(19) United States
(12) Patent Application Publication
(10) Pub. No.: US 2016/0030672 A1
(43) Pub. Date: Feb. 4, 2016

(54) Body Party and Diaphragm Materials for Medical Devices

(71) Applicant: SANOFI-AVENTIS DEUTSCHLAND GMBH, Frankfurt am Main (DE)

(72) Inventors: Markus Manderscheid, Frankfurt am Main (DE); James Alexander Davies, Warwickshire (GB)

(21) Appl. No.: 14/775,672

(22) PCT Filed: Mar. 13, 2014
(86) PCT No.: PCT/EP2014/054932

§ 371 Date: Sep. 12, 2015

(30) Foreign Application Priority Data
Mar. 15, 2013 (EP) 13159322.0

(51) Int. Cl. A61M 5/24; A61M 5/20; A61M 39/24

(52) U.S. Cl. CPC A61M 5/2448; A61M 39/24; A61M 5/2066; A61M 2039/2433; A61M 2005/3128

(57) ABSTRACT

The present invention inter alia relates to an apparatus. The apparatus comprises a body part, wherein the body part is made from a cyclic olefin polymer material or a cyclic olefin copolymer material or a mixture therefrom, and a diaphragm valve with a diaphragm, wherein at least a surface portion of the diaphragm is made from a diaphragm material, wherein said diaphragm material is a fluoroelastomer material or a perfluoroelastomer material or a mixture therefrom.

### CROSS REFERENCE TO RELATED APPLICATIONS

The present application is a U.S. National Phase Application pursuant to 35 U.S.C. §371 of International Application No. PCT/EP2014/054932 filed Mar. 13, 2014, which claims priority to European Patent Application No. 13159322.0 filed Mar. 15, 2013. The entire disclosure contents of these applications are herewith incorporated by reference into the present application.

### FIELD OF INVENTION

The present patent application inter-alia relates to medical devices for delivering at least two drug agents from separate reservoirs. Such drug agents may comprise a first and a second medicament. The medical device includes a dose setting mechanism for delivering the drug agents automatically or manually by the user.

### BACKGROUND

The medical device can be an injector, for example a hand-held injector, especially a pen-type injector, that is an injector of the kind that provides for administration by injection of medicinal products from one or more multidose cartridges. In particular, the present invention relates to such injectors where a user may set the dose.

The drug agents may be contained in two or more multiple dose reservoirs, containers or packages, each containing independent (single drug compound) or pre-mixed (co-formulated multiple drug compounds) drug agents.

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

### SUMMARY

For example, in some cases it may be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device. The proposed drug delivery device provides separate storage containers or cartridge retainers for two or more active drug agents. These active drug agents are then combined and/or delivered to the patient during a single delivery procedure. These active agents may be administered together in a combined dose or alternatively, these active agents may be combined in a sequential manner, one after the other.

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with a primary reservoir and with a secondary reservoir of medicament containing at least one drug agent. The drug dispense interface can be a type of outlet that allows the two or more medicaments to exit the system and be delivered to the patient.

The combination of compounds from separate reservoirs can be delivered to the body via a double-ended needle assembly. This provides a combination drug injection system that, from a user's perspective, achieves drug delivery in a manner that closely matches the currently available injection devices that use standard needle assemblies. One possible delivery procedure may involve the following steps:

1. Attach a dispense interface to a distal end of the electro-mechanical injection device. The dispense interface comprises a first and a second proximal needle. The first and second needles pierce a first reservoir containing a primary compound and a second reservoir containing a secondary compound, respectively.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable. Alternatively, the user can dial or set a desired dose of the secondary compound.

5. Optionally, after the second dose has been set, the device may be placed in an armed condition. The optional armed condition may be achieved by pressing and/or holding an “OK” or an “Arm” button on a control panel. The armed condition may be provided for a predefined period of time during which the device can be used to dispense the combined dose.

6. Then, the user will insert or apply the distal end of the dose dispenser (e.g. a double ended needle assembly) into the desired injection site. The dose of the combination of the primary compound and the secondary compound (and potentially a third medicament) is administered by activating an injection user interface (e.g. an injection button).

Both medicaments may be delivered via one injection needle or dose dispenser and in one injection step. This offers a convenient benefit to the user in terms of reduced user steps compared to administering two separate injections.

To prevent cross contamination and back flow of the first and second medicaments contained in the first and second reservoirs, respectively, the dispense interface may comprise a valve arrangement, preferably a small-size valve arrangement.

Preferably diaphragm valves are used in this valve arrangement, because diaphragm valves inter-alia have a low opening pressure threshold, give minimal resistance to flow when open and seal effectively against back pressure. For instance, in such a dispense interface, the opening pressure threshold of the diaphragm valve is preferably low to prevent leakage of the fluid connections to the medical device and/or the dose dispenser.

Furthermore, diaphragm valves can be designed to be very small.

The diaphragm of these diaphragm valves is made from soft materials such as rubbery-elastic materials like thermoplastic elastomers or liquid silicone rubbers. These soft materials usually contain plasticizers to provide for the necessary flexibility. For instance, this flexibility may inter-alia be necessary to provide a diaphragm valve having a low opening pressure threshold, giving minimal resistance to flow when open and/or sealing effectively against back pressure. However, soft materials containing plasticizers such as thermoplastic elastomers or liquid silicone rubbers are problematic in terms of biocompatibility. For instance, plasticizers contained in injection-mouldable soft materials are specifically problematic in terms of biocompatibility.

Typical plasticizers are for example phthalate-based plasticizers like bis(2-ethylhexyl) phthalate or diisooctyl phthalate, trimellitates like trimethyl trimellitate, or adipate-based plasticizers like bis(2-ethylhexyl)adipate.

Since diaphragms may at least partially be in (permanent) contact with the medicament, they are preferably made from biocompatible materials. For instance, plasticizers contained in a diaphragm may contaminate the medicament.

Soft materials containing plasticizers are also problematic in terms of durability. For instance, non-polar plasticizers typically used in a thermoplastic elastomer material have been found to leache from the thermoplastic elastomer material and to diffuse into body parts of the drug dispenser like, for example, the outside housing. Body parts are typically made from stiff rigid materials. The non-polar plasticizer diffusing into the body part material makes the material softer and may cause significant deformation, especially where the thermoplastic elastomer material comes into contact with the body part material. The deformation of a body part may deteriorate the functioning of the device, especially the functioning of the diaphragm valve, leading to defects in liquid tightness, and therewith cause a shorter lifetime of the device.

The use of plasticizer-free thermoplastic elastomer materials might prevent such deformations. However, such materials usually do not have suitable compression set properties.

The compression set of a material is the permanent deformation remaining when a force, that was applied to it, is removed. The compression set can be measured, for example, by test method B according to industrial standard ASTM D395. In this test a specimen of the material under testing is compressed by 25% for a specified time at a specified temperature. The compression set is taken as the percentage of the original deflection after the material is allowed to recover at standard conditions for 30 minutes.

Plasticizer-free thermoplastic elastomer materials have a rather high compression set property. Even after a short time in a stressed state, such a material does not fully return to its original shape. In a diaphragm valve with a diaphragm of such a material the closing pressure and the sealing pressure is not maintained, especially after a long shelf time of the device. These materials therefore cause a short lifetime of the device as well.

Though it might be possible for example to provide for a metal spring to keep up the elastic properties within the diaphragm valve, this may cause additional problems in terms of assembly, drug compatibility, drug resistance of the metal or the like.

Soft materials commonly used for diaphragms often do not have a good chemical resistance. In medical devices, like drug delivery devices, diaphragms of diaphragm valves are often in contact with constituents of drugs which may be chemically aggressive. For instance, m-cresol is a commonly used preservative in insulin based drugs. As most soft materials are not resistant to m-cresol, the m-cresol in the drug may corrode the diaphragm material which may result in defects in liquid tightness and short lift-time of the diaphragm valve and/or the apparatus.

Therefore, the present invention inter-alia faces the technical problem of providing a biocompatible diaphragm for a diaphragm valve such as a diaphragm valve used in a valve arrangement of a dispense interface, which improves the life-time of the diaphragm valve and/or the dispense interface.

According to the present invention, an apparatus comprises a body part, wherein the body part is made from a cyclic olefin polymer material or a cyclic olefin copolymer material or a mixture therefrom, and a diaphragm valve with a diaphragm, wherein at least an outer part of the diaphragm is made from a diaphragm material, wherein said diaphragm material is a fluoroelastomer material or a perfluoroelastomer material or a mixture therefrom.

The apparatus may be a drug delivery device such as a medical device configured to eject a drug agent (e.g. a dose of a medicament) such as an infusion device or an injection device, for instance an insulin injection pen. Injection devices may be used either by medical personnel or by patients themselves. As an example, type-1 and type-2 diabetes may be treated by patients themselves by injection of insulin doses, for example once or several times per day.

For instance, the apparatus is a medical device configured to eject at least two drug agents from separate reservoirs comprising a first and a second medicament, respectively, but it is not limited thereto. Alternatively, the medical device is for instance a conventional medical device configured to eject a drug agent from a single reservoir such as Applicant's Solostar® insulin injection pen.

Alternatively, the apparatus may be a disposable part attachable to a medical device such as a drug delivery device. For instance, the apparatus is a dispense interface attachable to a medical device configured to eject a drug agent. A dispense interface may be configured to be in fluid communication with at least one reservoir of the medical device containing at least one medicament. For instance, the dispense interface is a type of outlet that allows the at least one medicament to exit the medical device.

The diaphragm valve may comprise a valve body and the diaphragm. The valve body may comprise a valve chamber and an inlet and an outlet port of the valve chamber. For instance, the inlet port is arranged at a sidewall of the valve chamber. The inlet port may be in fluid communication with a reservoir containing a fluid such as a reservoir of the medical device. The outlet port of the valve chamber may be arranged at a convex protrusion of the valve chamber, wherein the convex protrusion extends into the valve chamber. The outlet port may be in fluid communication with an outlet of the dispense interface. For instance, the inlet and/or the outlet port are respectively one of an end of a pierce needle, a start point of a fluid groove, a septum or the like.

The diaphragm may be arranged in the valve chamber such that the diaphragm provides a fluid seal between the inlet port and the outlet port. For instance, a protrusion at the center of the diaphragm is arranged in a valve cavity positioned in the center of the convex protrusion and the diaphragm separates the inlet port of the valve chamber from the outlet port of the valve chamber. In such an arrangement, the diaphragm provides a fluid seal between the inlet port and the outlet port, the diaphragm valve is closed.

For instance, the area of the surface of the convex protrusion oriented towards the diaphragm may be not less than the area of the surface of the diaphragm oriented towards the convex protrusion such that, if the diaphragm is inverted (e.g. put over the convex protrusion) and resides along the convex protrusion, the diaphragm valve does at least partially not cover the outlet port and does not provide a fluid seal between the inlet port and the outlet port, the diaphragm valve is opened and the diaphragm allows fluid flow from the inlet port to the outlet port.

As an example, the valve chamber may at least partially be formed from a circular recess and a cover. The inlet port may be arranged at this circular recess; and the outlet port and the convex protrusion is for instance arranged at this cover. The diaphragm or a rim of the diaphragm for instance resides on a circular set-back of the recess such that the diaphragm provides a fluid seal between the inlet port and the outlet port such that the valve is closed. For instance, the valve cavity of the convex protrusion of the cover may at least partially fix the center of the diaphragm in the valve chamber, for instance it presses the diaphragm or the rim of the diaphragm on the circular set-back of the recess such that the diaphragm initially sits in a pre-stressed state in the valve chamber.

If (fluidic) pressure is applied upon a surface of the diaphragm oriented towards the inlet port of the valve chamber, the diaphragm may change from the pre-stressed state to an even more stressed state (which is below referred to as stressed state), alternatively the diaphragm may change from an un-stressed state to the stressed state; and, if an opening pressure threshold is overcome, the diaphragm inverts and resides along the convex protrusion. The (fluidic) pressure may be increased by increasing the pressure in a fluid reservoir being in fluid communication with the inlet port. In the stressed state, the diaphragm may accordingly enable a fluid flow from the inlet port to the outlet port. The opening pressure threshold may advantageously correspond to a pressure of about 25 mbar to 200 mbar such as 50 mbar, 100 mbar or 150 mbar, but is not limited thereto.

However, if the (fluidic) pressure falls below a closing pressure threshold, the diaphragm may return to the initial shape and, accordingly, disables the fluid flow from the inlet port to the outlet port. Back pressure applied on the surface of the diaphragm oriented towards the outlet port of the valve chamber may for instance amplify the returning of the diaphragm to the initial-state.

The opening pressure threshold may relate to the stiffness of the diaphragm. Stiffness is to be understood to relate to the physical property of the diaphragm to resist to a (elastic) deformation by a stress.

The closing pressure threshold may relate to the elastic counterforce of the diaphragm in response to the inversion and/or to the elasticity of the diaphragm. Elasticity is to be understood to relate to the physical property of the diaphragm to return to the initial shape after a stress which made it (elastically) deform is (partially) removed.

The closing pressure threshold may be equal to the opening pressure threshold.

According to the present invention, the body part is made from a cyclic olefin polymer material or a cyclic olefin copolymer material or a mixture therefrom.

Other stiff rigid materials like polycarbonates (PC) or Polymethyl methacrylate (PMMA) often show significant water absorption and are therefore less suitable for water and water soluble medicaments. In particular, these materials may tend to dimensional instability due to water absorption.

Cyclic olefin polymer materials, cyclic olefin copolymer materials or mixtures therefrom have good properties regarding their resistance to water and water soluble medicaments. Especially, they have low water absorption and high water vapor barrier, i.e. good moisture barrier properties, and are dimensionally stable.

Cyclic olefin polymer materials, cyclic olefin copolymer materials or mixtures therefrom also have a good capability of being laser welded which facilitates the production process and allows the ullage of a drug in the apparatus, especially in a dispense interface, to be minimized. For example, the body part made from a cyclic olefin polymer material or a cyclic olefin copolymer material or a mixture therefrom, may also be doped with a laser welding additive to increase the body part's sensitivity to laser light and thus to facilitate laser welding.

The use of cyclic olefin polymer materials, cyclic olefin copolymer materials or mixtures therefrom is inter-alia advantageous in order to provide a material suitable for contact with insulins and to improve the lifetime of the apparatus.

Examples for cyclic olefin polymer materials, cyclic olefin copolymer materials or mixtures therefrom are: Zeonex® provided by Zeon Chemicals L.P. or Topas® COC provided by Topas Advanced Polymers GmbH.

However, the good properties of the body part made of cyclic olefin polymer materials, cyclic olefin copolymer materials or mixtures therefrom may be foiled by materials often used for the diaphragm of the diaphragm valve. For instance, plasticizers may leach from diaphragms made of plasticizer-containing thermoplastic elastomers and soften the cyclic olefin polymer or copolymer material of the body part leading to deformations of the same.

According to the present invention, at least a surface portion of the diaphragm is made from a diaphragm material, wherein said diaphragm material is a fluoroelastomer material or a perfluoroelastomer material or a mixture therefrom.

A surface portion of the diaphragm is understood to mean a portion of the diaphragm which forms at least a part of the surface of the diaphragm. For instance, the surface portion of the diaphragm may be the diaphragm in its entirety, i.e. the diaphragm may be entirely made of the diaphragm material. The surface portion also may be a partial or overall coating. Especially, the diaphragm may comprise a core, which may be made from a core material different to the diaphragm material, and a coating, which coating is made from the diaphragm material. An example for a diaphragm material that can be used to coat a core of a diaphragm is FluroTec® provided by West Pharmaceutical Services, Inc.

Fluoroelastomer materials or perfluoroelastomer materials or mixtures therefrom are thermoset materials. Production of these materials involves a vulcanization process in which cross-links between the polymer chains are built up. The cross-linked composition of these materials yields good elasticity and a low compression set. Especially, it is not necessary to add plasticizers to achieve the good elasticity and compression set properties which are suitable properties of materials for long-life diaphragms of diaphragm valves.

A diaphragm made of or at least partially made of said diaphragm material may contain less plasticizer than thermoplastic elastomers or may even be plasticizer-free. Thus, the leaching of plasticizers from the diaphragm and the softening of the body part material causing deformation can be reduced or even prevented. The fluoroelastomer or perfluoroelastomer material or mixtures therefrom used as diaphragm material therefore has good compatibility with the cyclic olefin polymer materials, cyclic olefin copolymer materials or mixtures therefrom used for the body part of the apparatus.

In addition, fluoroelastomer materials or perfluoroelastomer materials or mixtures therefrom have very good chemical resistance including resistance to, for example, m-cresol, which is commonly used as a preservative in insulin based drugs. The diaphragm material is therefore suitable to be used for a diaphragm in an apparatus, in which the diaphragm may have contact with aggressive chemicals, in particular chemically aggressive drug constituents like m-cresol.

The use of fluoroelastomer materials or perfluoroelastomer materials or mixtures therefrom as diaphragm material is inter-alia advantageous in order to improve the life-time of the diaphragm valve and/or the apparatus.

Examples for fluoroelastomer materials or perfluoroelastomer materials or mixtures therefrom are: Viton® provided by DuPont™ Elastomers, Perlast® provided by C. Otto Gehrckens GmbH & Co. KG, Resifluor™ provided by Trelleborg AB, or FluroTec® provided by West Pharmaceutical Services, Inc.

According to the present invention, two different materials are selected for a body part and for a diaphragm of a diaphragm valve of an apparatus: a cyclic olefin polymer material or a cyclic olefin copolymer material or a mixture therefrom is selected for the body part and a fluoroelastomer material or a perfluoroelastomer material or a mixture therefrom is selected as diaphragm material for the diaphragm or at least a surface portion thereof. This specific combination of materials has the synergetic effect that the apparatus has a prolonged life-time. One the one hand, it may be advantageous to select a dimensionally stable material for the body part and a persistent flexible material for the diaphragm to improve the life-times of these individual components. An inappropriate combination of two such materials may however be detrimental to the life-time of the whole apparatus. The combination of materials according to the present invention, on the other hand, has the further advantageous effect that the two components do not mutually derogate their life-times so that the whole apparatus has a prolonged life-time.

In the following, features and embodiments (exhibiting further features) of the present invention will be described, which are understood to apply to the apparatus as described above. These single features/embodiments are considered to be exemplary and non-limiting, and to be respectively combinable independently from other disclosed features/embodiments of the apparatus as described above. Nevertheless, these features/embodiments shall also be considered to be disclosed in all possible combinations with each other and with the apparatus as described above.

According to an embodiment of the present invention said diaphragm material has a content of plasticizers of less than 10% by weight, preferably of less than 5% by weight, more preferably of less than 1% by weight, especially of less than 0.5% by weight. The low plasticizer content of the diaphragm material reduces leaching of plasticizer from the diaphragm material and diffusion into the body part material. This embodiment is inter-alia advantageous in order to increase the life-time of the diaphragm valve and/or the apparatus.

According to an embodiment of the present invention said diaphragm material is a plasticizer-free fluoroelastomer material or a plasticizer-free perfluoroelastomer material or a mixture therefrom. Something is understood to be plasticizer-free if its plasticizer content is less than 0.1% by weight, preferably less than 0.01% by weight, more preferably less than 0.001% by weight, especially less than 0.0001% by weight. A plasticizer-free diaphragm material prevents leaching of plasticizer from the diaphragm material and diffusion into the body part material. This embodiment is inter-alia advantageous in order to increase the life-time of the diaphragm valve and/or the apparatus.

According to an embodiment of the present invention the diaphragm is entirely made from said diaphragm material. A diaphragm entirely made from the diaphragm material may contain very little or even no plasticizer at all. Leaching of plasticizer from the whole diaphragm and diffusion into the body part material may therefore be reduced or prevented. This embodiment is inter-alia advantageous in order to increase the life-time of the diaphragm valve and/or the apparatus.

According to an embodiment of the present invention said diaphragm material is a FKM-ML-X18 material. FKM-ML-X18 is a fluoroelastomer material which is commercially available and which is basically plasticizer-free. Experiments have shown that FKM ML-X18 has very good insulin biocompatibility. Furthermore, FKM ML-X18 provides suitable low compression set properties to achieve sufficient closing pressure and sealing pressure for a diaphragm valve over a long lifetime. FKM ML-X18 may provide these properties even without the addition of any plasticizers. In particular, the FKM ML-X18 is plasticizer-free. This embodiment is inter-alia advantageous in order to provide a diaphragm made from a material which has a good compatibility with insulin drugs.

According to an embodiment of the present invention the diaphragm valve is accommodated within a housing which is at least partially formed by the body part. For example, the housing may be configured as the outer housing of the apparatus or part of it. The housing may also be configured as an inner housing of the apparatus which itself is accommodated within another outer housing of the apparatus. This embodiment is inter-alia advantageous in order to provide for a dimensionally stable housing of the apparatus with which the ullage of medicaments may be minimized.

According to an embodiment of the present invention the body part is in direct contact with the diaphragm. For example, the body may be configured as part of the diaphragm valve or as a part bearing the diaphragm valve, in particular bearing the diaphragm. Diaphragms made of plasticizer-containing thermoplastic elastomers can in particular soften body parts which are in direct contact with the diaphragm and thus lead to deformations. Body parts which are in direct contact with the diaphragm are however usually parts of particular importance for the functioning and liquid tightness of the diaphragm valve and/or the apparatus. This embodiment is therefore inter-alia advantageous in order to provide for long-life functioning of the diaphragm valve.

According to an embodiment of the present invention the apparatus comprises a first body part and a second body part, said second body part being configured as cover part, wherein said first body part and said second body part are configured to at least partially form a fluid channel between a surface of said first body part and a surface of said second body part, and wherein the first and/or the second body part is/are made from a cyclic olefin polymer material or a cyclic olefin copolymer material or a mixture therefrom.

Apparatuses comprising a diaphragm valve usually also comprise fluid channels, in particular to guide a fluid to and/or from the diaphragm valve. The first body part and the second body part according to this embodiment can be simply manufactured, for instance by moulding such as injection moulding. By joining the first body part and the second body part after manufacturing thereof, it is possible to form a joined part having fluid channels with a high length-to-diameter ratio and/or a complex geometry and/or tight tolerances. This embodiment is therefore inter-alia advantageous to allow simple manufacturing of the apparatus.

According to an embodiment of the present invention said first body part comprises a recess and a first body part reservoir and/or a second body part reservoir, wherein said fluid channel provides a fluid connection from said first body part reservoir and/or said second body part reservoir to said recess, and wherein at least one of said first and/or second body part reservoir is configured to receive the diaphragm valve.

For example, the recess defines an outlet of the fluid channel, and the first and second body part reservoir define a first and second inlet of the fluid channel, respectively.

The recess may be configured to at least partially receive one proximal end of a needle of a dose dispenser and to reside in fluid connection with the needle of the dose dispenser. For instance, the dose dispenser is a standard needle assembly or a double-ended needle assembly. For instance, a distal end of the needle is inserted into a desired injection site before an injection.

The first and the second body part reservoir may be configured to be in fluid connection with a respective fluid reservoir. For instance, a first and second piercing needle ends in the first and second body part reservoir, respectively. In particular, the first and second piercing needles may be configured to pierce a septum of a respective fluid reservoir such as a medicament reservoir, cartridge and/or a container, to reside in fluid connection with the fluid reservoir and provide a fluid communication between the respective fluid reservoir and the first and second body part reservoir. The first and second body part reservoir may at least partially form a valve chamber, for instance the first and second body part are configured to at least partially receive the diaphragm of the diaphragm valve.

For instance, the apparatus is a dispense interface providing a fluid connection from a first and second fluid reservoir of a drug delivery device to a needle assembly of a dispense interface, for instance the apparatus provides the fluid connection via the first and second body part reservoir, the fluid channel and the recess. For instance, the fluids from the first and second fluid reservoir enter the fluid channel via a respective first and second diaphragm valve at least partially arranged in the respective first and second body part reservoir.

In particular at least a portion of a diaphragm of the first diaphragm valve and/or a diaphragm of the second diaphragm valve is made from the diaphragm material, which is a fluoroelastomer or a perfluoroelastomer material, or a mixture therefrom.

According to an embodiment of the present invention at least one of said first and/or second body part reservoir is configured to receive the diaphragm valve such that the diaphragm valve is at least substantially arranged in a first plane parallel to a longitudinal axis of said apparatus. For instance, the first and second body part reservoir are configured to receive a first and second diaphragm valve, respectively, such that the first and second diaphragm valve are at least substantially arranged in a first plane parallel to a longitudinal axis of the apparatus.

Something may be understood to be arranged at least substantially arranged in a plane, if it is cut by the plane and/or a longitudinal axis (e.g. an axis along the direction of the largest extension) thereof is coplanar or at least substantially coplanar with the plane. A longitudinal axis is for instance substantially coplanar with a plane, if the longitudinal axis includes an angle of less than 30°, preferably less than 10° with the plane.

As described above, the first and second body part reservoir may at least partially form a valve chamber for a first and second diaphragm valve. For instance, a first and second diaphragm valve may at least partially be received in the first and second body part reservoir, respectively.

The diaphragm valve is at least substantially arranged in the first plane. For instance, the diaphragm of the diaphragm valve is at least partially received in one of the first and/or second body part reservoir such that the first plane cuts the diaphragm valve. For instance, a longitudinal axis of the diaphragm valve is at least substantially coplanar with the first plane. For instance, a symmetry axis, such as a rotational axis of the diaphragm valve is angled to the first plane, for instance the symmetry axis includes an angle equal to or greater than 60°, preferably 80° with the first plane. In particular, the symmetry axis may be perpendicular to the first plane and/or to a surface of the diaphragm valve facing the first body part and/or the second body part.

The diaphragm valve may be at least substantially flat. Something may be understood to be substantially flat, if the diameter thereof in direction of the longitudinal axis is at least twice of the thickness thereof in perpendicular direction of the longitudinal axis. For instance, the diaphragm of the diaphragm valve has a generally convex shape. A symmetry axis of the generally convex shaped diaphragm may be a centerline running through the apex thereof; the longitudinal axis of the generally convex shaped diaphragm may be perpendicular to the symmetry axis.

The longitudinal axis of the apparatus may be a vertical centerline of the apparatus such that, for instance the first plane is a vertical plane. For instance, the first plane is at least partially coplanar with the joining area of the surfaces of the first body part and the second body part. The first plane may be spaced from the longitudinal axis of the apparatus. For instance, the first plane is spaced from, but parallel to a symmetry plane of the apparatus. Alternatively or additionally, the first plane may be perpendicular to a symmetry plane of the apparatus. The first plane may laterally cut the apparatus, for instance the first plane is a lateral plane parallel to a symmetry plane of the apparatus. For instance, a symmetry plane of the apparatus is coplanar with the longitudinal axis of the apparatus. For instance, the first and/or second body part reservoir and/or the first and/or second piercing needle may be arranged in a symmetry plane of the apparatus. The symmetry plane may cut the first and second body part reservoir. The symmetry plane may be coplanar with the longitudinal axis of the first and second piercing needle.

This embodiment is inter alia advantageous to allow arranging the first body part and the second body part such that the fluid channel may be formed at least partially in a lateral plane parallel to the first plane. Furthermore, it is inter alia advantageous to allow the first body part and the second body part to be joined in a lateral plane parallel to the first plane, for instance by laser welding by a laser laterally positioned, for instance angled or perpendicular to the first plane. Laterally positioning the laser is for instance advantageous, because from such a lateral position the laser may only need to pass through the second body part which may have an at least substantially uniform thickness at the joining area. In contrast to this, from a longitudinal position, for instance perpendicular to the first plane, the laser may typically need to pass through additional components at least partially covering the second body part resulting in undesired scattering and attenuation of the laser beam. In other words, this embodiment is inter alia advantageous to allow the first body part and the second body part to be joined at a joining area of surfaces of the first body part and the second body part which is easily accessible by a laser.

According to an embodiment of the present invention, at least one of the first body part and the second body part comprises a groove arrangement, the groove arrangement arranged in the surface of the at least one of the first body part and the second body part.

For instance, the groove arrangement is a fluid groove arrangement. The groove arrangement may comprise any number of grooves, which may be any indentations on the surface of the respective part which permits the passing of fluid along the surface thereof.

According to an embodiment of the present invention said first body part defines an inner body of said apparatus and said second body part defines a manifold of said apparatus.

According to an embodiment of the present invention said second body part comprises at least a first valve cavity provided along its top surface, the first valve cavity is shaped for receiving a protrusion of the diaphragm of the diaphragm valve and the first valve cavity is positioned near an apex of the protrusion of the diaphragm.

According to an embodiment of the present invention, surfaces of the first body part and the second body part are at least substantially flat at a joining area, for instance the joining area described above. For instance, the joining area of the surfaces extends along an outer edge of at least one of the first body part and the second body part, for instance an outer edge of the surface of the second body part.

The joining area of the surfaces may be an at least substantially flat circumferential area which may enclose a 3-dimensionally structured centric area. For instance, the (3-dimensional) groove arrangement is entirely arranged in the centric area such that the surfaces joined at the joining area may seal the entire groove arrangement. This embodiment is inter alia advantageous to allow a comprehensive and tight sealing of the fluid channel (e.g. the groove arrangement) by the surfaces of the first body part and the second body part at the joining area.

According to an embodiment of the present invention, the surfaces of the first body part and the second body part are joined by laser welding at a joining area, for instance the joining area described above. A cyclic olefin polymer or cyclic olefin copolymer material or a mixture therefrom, which is used for the first and/or the second body part, has very good laser-welding properties so that a tight and durable laser weld can be achieved.

In particular, the laser welding may be a laser-transmission-welding. For instance, the laser welding track (e.g. a laser welding line) defines the joining area of the surfaces of the first body part and the second body part. The laser welding track may be a closed track on the surfaces of the body part and the second body part. For instance, the laser welding track extends along an outer edge of the surface of the second body part.

The welding laser may be a gas laser or a solid state laser such as a diode laser. Preferably, the welding laser may be a Pulsed Fiber Laser. The wavelength of the welding laser may be between 100 nm and 10 μm, preferably between 900 nm and 1100 nm, in particular one of 1962 nm, 1062 nm+/−3 nm or 1062 nm+/−10 nm.

This embodiment is inter alia advantageous to allow a comprehensive and tight sealing of the fluid channel. Especially, laser welding joints are mechanically highly stressable and pressure-sealed. Often the laser joints reach the strength of the basic materials. The laser joining also yields very good surface qualities, in particular micro particles, glue residues or increased surface roughness are at a low level or even not present.

Laser welding is also advantageous from a procedural point of view. The laser welding process is highly flexible and can be adapted easily to design changes and the laser pattern. Moreover, the choice of the welding laser wavelength allows selecting the reactions taking place in the welded parts during welding. Since laser welding is a non-contact process, insertion of mechanical and/or thermal energy into the parts to be welded is minimal. Sensitive parts, like the diaphragms of a diaphragm valve, remain unaffected.

According to an embodiment of the present invention the apparatus is a medical device or a part of a medical device.

According to an embodiment of the present invention said diaphragm valve is configured to enable fluid flow, if a fluidic pressure threshold is applied on said diaphragm valve.

According to an embodiment of the present invention the apparatus is a medical device configured to eject a medicament and the diaphragm valve is configured to control fluid communication of a medicament contained in a reservoir of the drug delivery device and a dose dispenser.

According to an embodiment of the present invention said apparatus comprises at least two of said diaphragm valves and the medical device respectively comprises at least two of said reservoirs.

### BRIEF DESCRIPTION OF THE DRAWINGS

These as well as other advantages of various aspects of the present invention will become apparent to those of ordinary skill in the art by reading the following detailed description, with appropriate reference to the accompanying drawings, in which:

FIG. 1 illustrates a perspective view of a delivery device with an end cap of the device removed;

FIG. 2 illustrates a perspective view of the delivery device distal end showing the cartridge;

FIG. 3 illustrates a perspective view of the delivery device illustrated in FIG. 1 or 2 with one cartridge retainer in an open position;

FIG. 4 illustrates a dispense interface and a dose dispenser that may be removably mounted on a distal end of the delivery device illustrated in FIG. 1;

FIG. 5 illustrates the dispense interface and the dose dispenser illustrated in FIG. 4 mounted on a distal end of the delivery device illustrated in FIG. 1;

FIG. 6 illustrates one arrangement of a needle assembly that may be mounted on a distal end of the delivery device;

FIG. 7 illustrates a perspective view of the dispense interface illustrated in FIG. 4;

FIG. 8 illustrates another perspective view of the dispense interface illustrated in FIG. 4;

FIG. 9 illustrates a cross-sectional view of the dispense interface illustrated in FIG. 4;

FIG. 10 illustrates an exploded view of the dispense interface illustrated in FIG. 4;

FIG. 11 illustrates a cross-sectional view of the dispense interface and needle assembly mounted onto a drug delivery device, such as the device illustrated in FIG. 1;

FIG. 12 illustrates a cross-sectional view of an alternative embodiment of a dispense interface;

FIG. 13 illustrates an exploded view of the alternative embodiment of a dispense interface illustrated in FIG. 12;

FIG. 14 illustrates a manifold of a dispense interface such as the alternative embodiment of the dispense interface illustrated in FIG. 12;

FIG. 15 illustrates a schematic cross-sectional view of diaphragm valves arranged between a manifold and an inner body of the dispense interface illustrated in FIG. 12 joined by laser welding;

FIG. 16 illustrates a cross-sectional view of a manifold and an inner body of a dispense interface such as the alternative embodiment of the dispense interface illustrated in FIG. 12 joined by laser welding; and

FIG. 17 illustrates a manifold and an inner body of a dispense interface such as the alternative embodiment of the dispense interface illustrated in FIG. 12 joined by laser welding.

### DETAILED DESCRIPTION

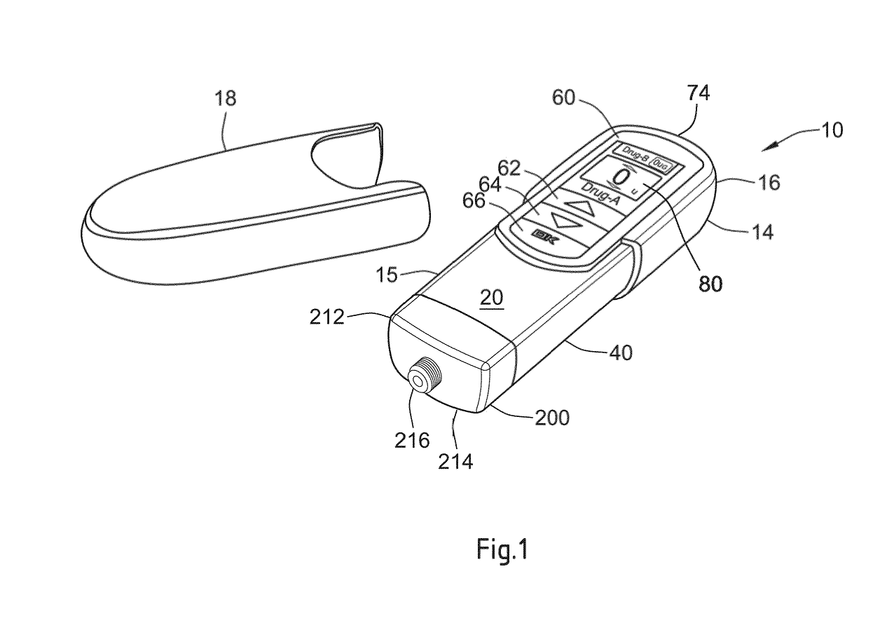

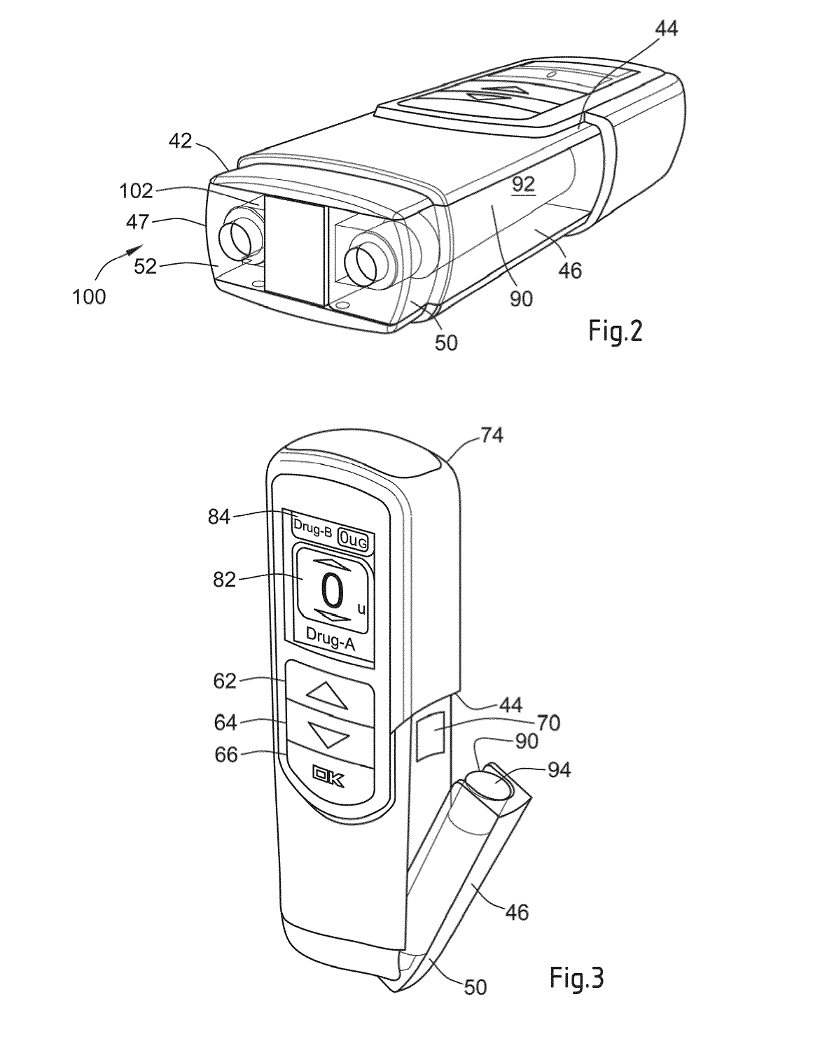

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

The drive train may exert a pressure on the bung of each cartridge, respectively, in order to expel the doses of the first and second medicaments. For example, a piston rod may push the bung of a cartridge forward a pre-determined amount for a single dose of medicament. When the cartridge is empty, the piston rod is retracted completely inside the main body 14, so that the empty cartridge can be removed and a new cartridge can be inserted.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1). The user interface of the drug delivery device may comprise additional buttons, such as a “menu” button, a “back” button, or a “light” button to switch on an illumination of the display.

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

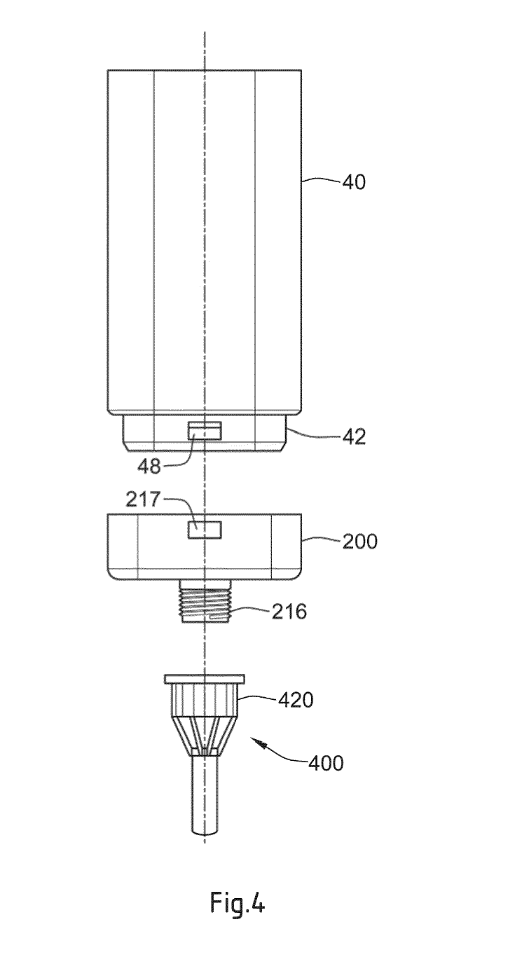

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and second cartridge retainers 50, 52 may be hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 can be coupled to the distal end of the cartridge holder 40. FIG. 4 illustrates a flat view of the dispense interface 200 unconnected to the distal end of the cartridge holder 40. A dose dispenser or needle assembly 400 that may be used with the interface 200 is also illustrated and is provided in a protective outer cap 420.

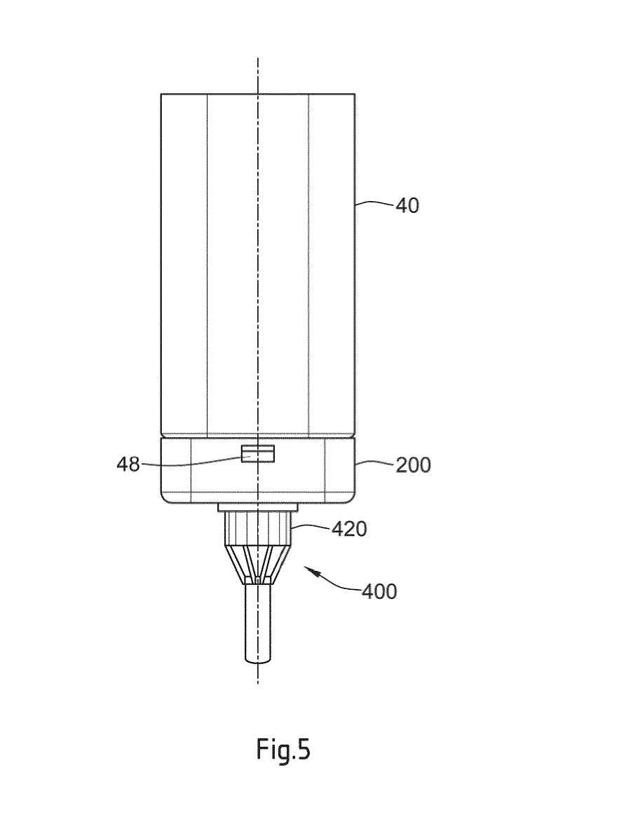

In FIG. 5, the dispense interface 200 illustrated in FIG. 4 is shown coupled to the cartridge holder 40. The axial attachment means 48 between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

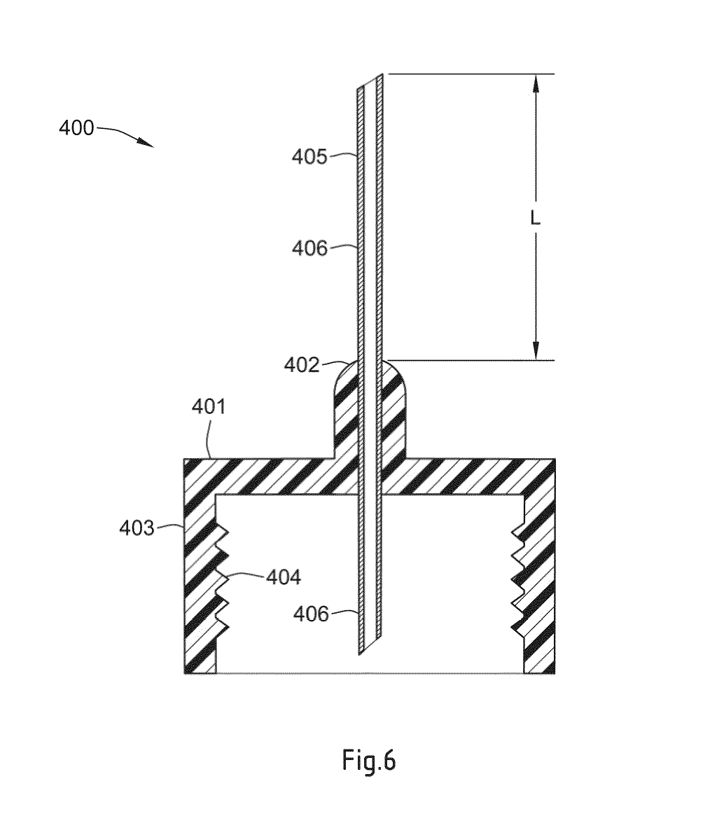

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 400 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 408 of the needle assembly 400 protrudes from an opposite side of the circular disc so that it is concentrically surrounded by the sleeve 403. In one needle assembly arrangement, the second or proximal piercing end 408 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

Referring now to FIGS. 4 to 11, one preferred arrangement of this interface 200 will now be discussed. In this one preferred arrangement, this interface 200 comprises:

a. a main outer body 210,

b. an first inner body 220,

c. a second inner body 230,

d. a first piercing needle 240,

e. a second piercing needle 250,

f. a valve seal 260, and

g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

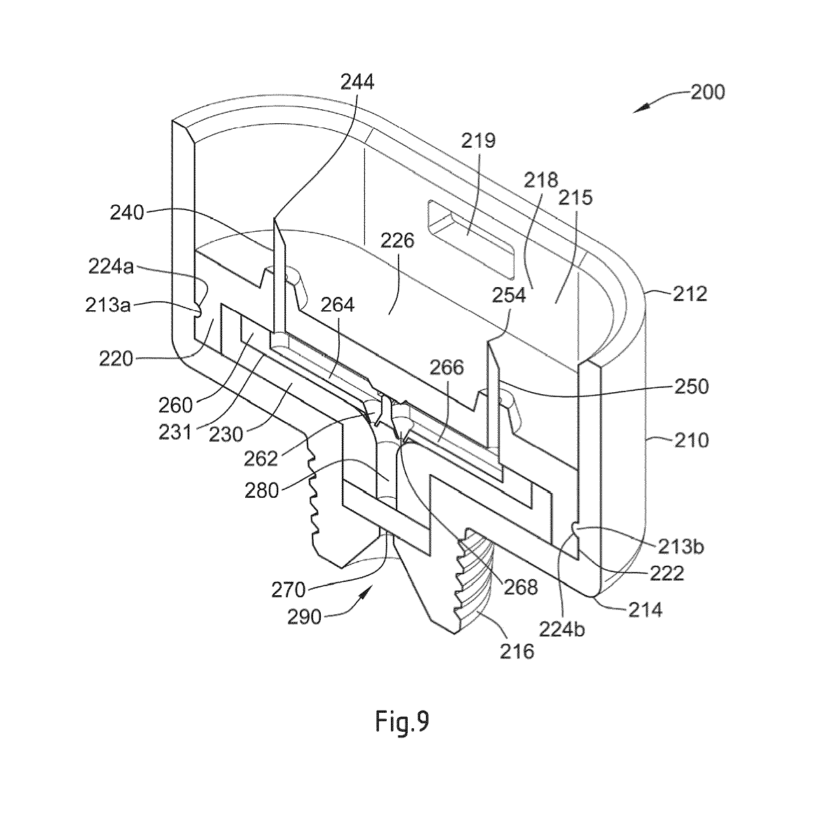

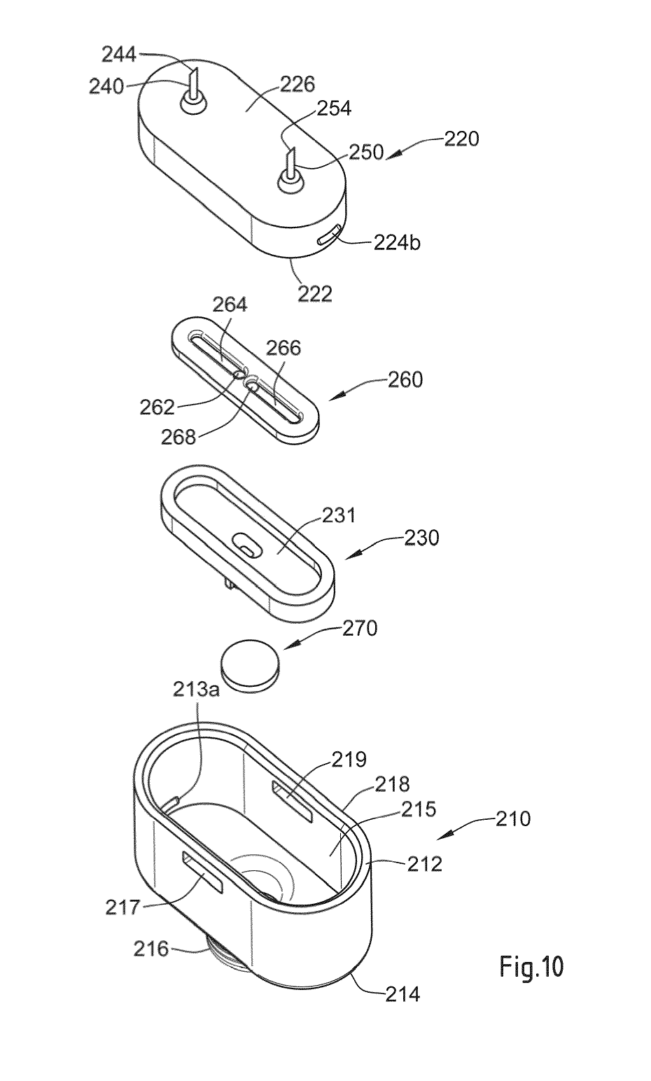

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

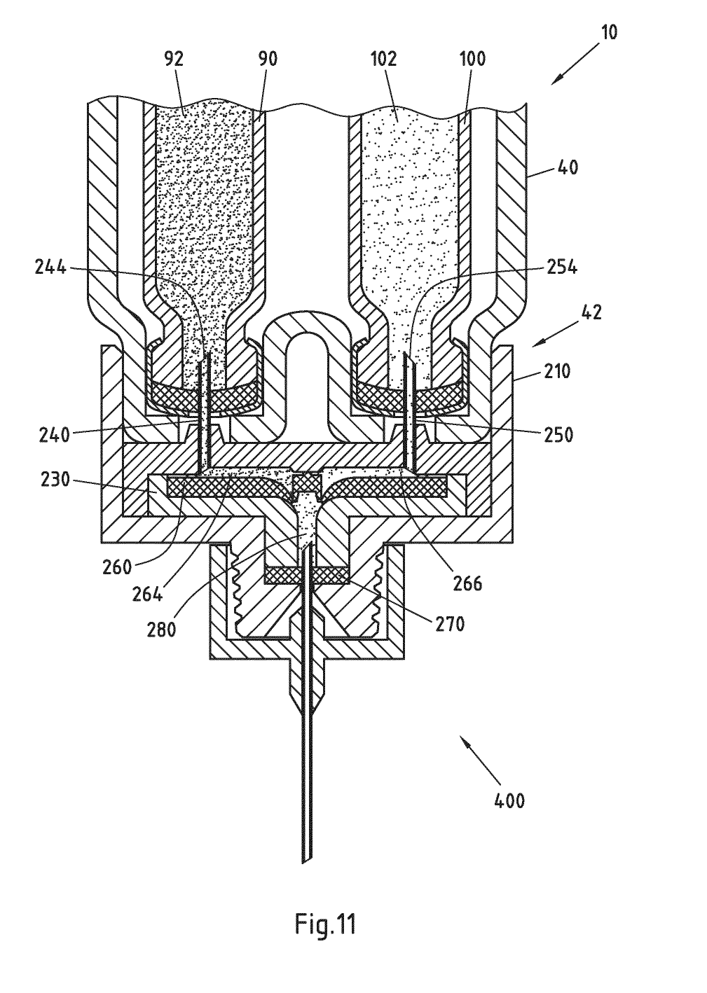

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

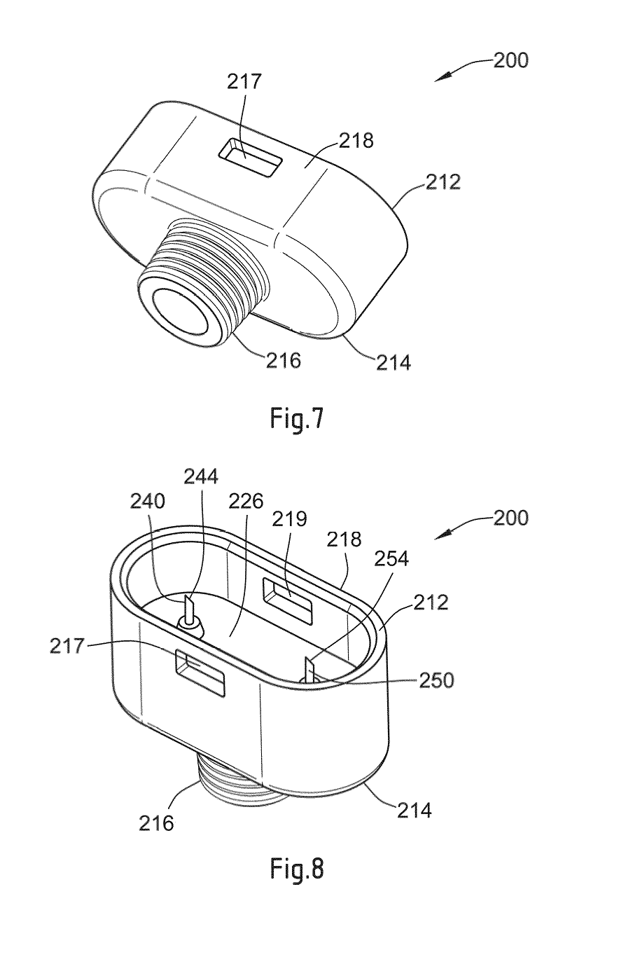

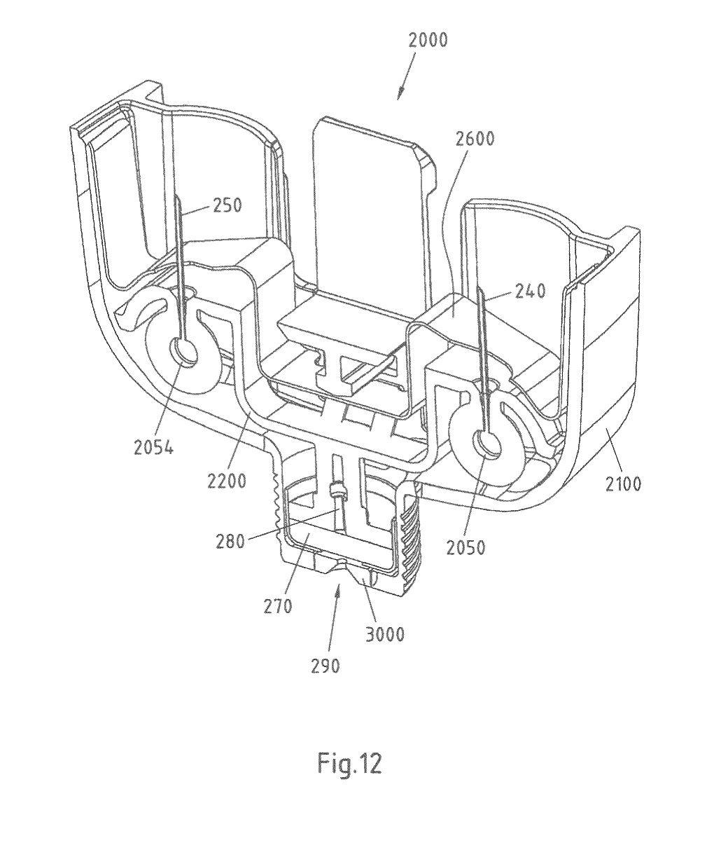

FIGS. 12 to 17 illustrate an embodiment of a dispense interface 2000 alternative to the embodiment of the dispense interface 200 illustrated in FIGS. 7 to 11. In FIGS. 12 to 17 the same reference signs as in FIGS. 7 to 11 are used for parts which may be similar. Furthermore, at this point, it is mainly referred to the above description of the embodiment of the dispense interface 200 illustrated in FIGS. 7 to 11 and, basically, the differences are described only.

As will now be discussed in greater detail, in one preferred arrangement, the dispense interface 2000 illustrated in FIGS. 12 to 17 comprises:

a. a main outer body 2100;

b. an inner body 2200;

c. a manifold 2300;

d. a first piercing needle 240;

e. a second piercing needle 250;

f. a lock-out spring 2600;

g. a first diaphragm valve 2700;

h. a second diaphragm valve 2750;

i. a ferrule 2800

j. an outer septum 270; and

k. a needle guide 3000.

One exemplary difference between the dispense interface 200 and the dispense interface 2000 is the outer shape. In particular, the dispense interface 2000 is attachable to a drug deliver device by axial attachment means as described above and at least partially insertable in the drug delivery device. For instance, once the dispense interface 2000 is attached to the distal end of the drug delivery device, the distal end of the main body of the drug delivery device covers a portion of the dispense interface 2000.

One further exemplary difference between the dispense interface 200 and the dispense interface 2000 is the manifold 2300, which resides on the inner body 2200 such that a “Y”-shaped fluid channel is formed between the facing surfaces of the manifold 2300 and the inner body 2200.

The function of the first and second diaphragm valve 2700, 2750 of the dispense interface 2200 may basically relate to the function of the first and second non return valve 262, 264 of the dispense interface 200. As described above, such a valve arrangement may for instance be constructed so as to prevent back flow and/or cross contamination of the first and second medicaments contained in the first and second reservoirs, respectively.

Furthermore, the dispense interface 2000 comprises a dispense interface lockout element in the form of a lockout spring 2600. One reason that a lock out member may be incorporated into a dispense interface, such as the interface 2000, is to ensure that once the dispense interface is removed from the drug delivery device, the dispense interface cannot be reattached and used a second time. Preventing re-attachment tends to ensure that medicament is not allowed to reside in the dispense interface 2000 indefinitely and contaminate the drug delivered to the patient.

The ferrule 2800 may basically serve for holding the outer septum 270; and the needle guide 3000 of the dispense interface 2000 may basically serve for centering a proximal end of a needle assembly before piercing the outer septum 270.

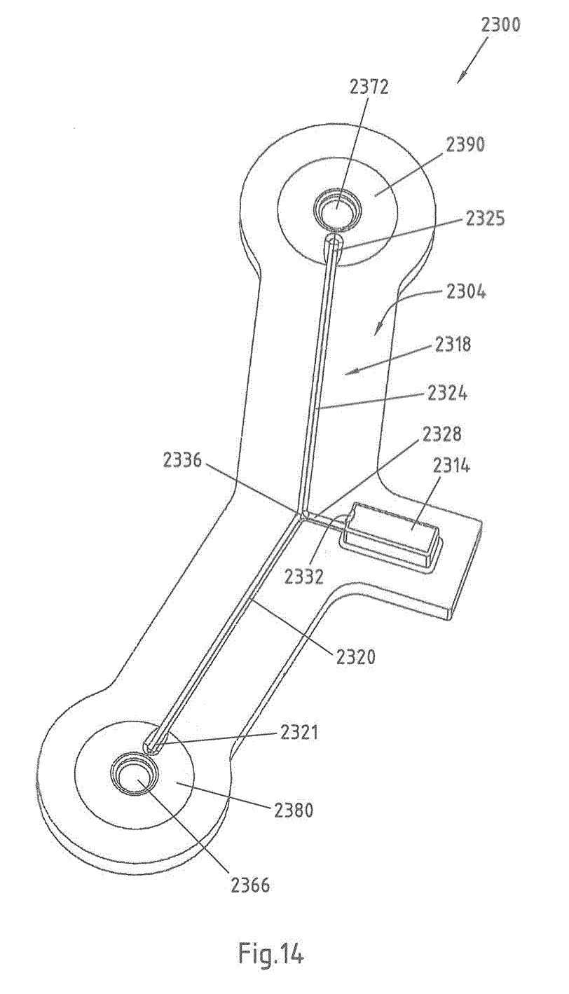

As illustrated in FIG. 14, the manifold 2300 comprises a first valve cavity 2366 and a second valve cavity 2372 provided along its top surface 2304. These cavities 2366, 2372 may be substantially flat and circular. The first valve cavity 2366 is configured to receive a circular protrusion 2710 of a first diaphragm 2700. Similarly, the second valve cavity 2372 is shaped for receiving a circular protrusion 2760 of a second diaphragm 2750.

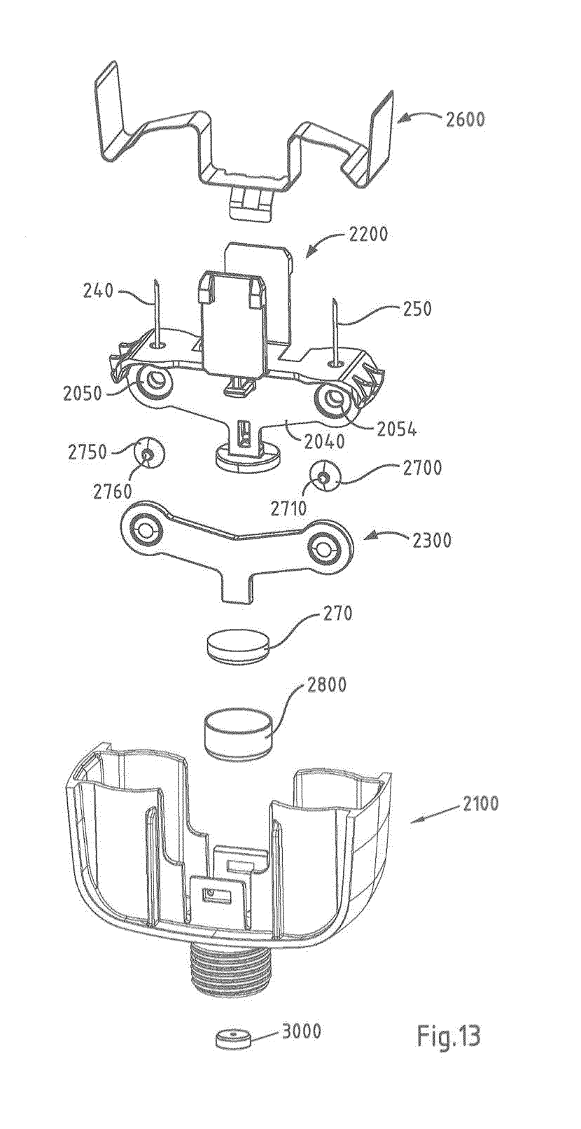

For example, in the exploded view illustrated in FIG. 13, alternative perspective views of both the first diaphragm 2700 and the second diaphragm 2750 are provided. As can be seen from this exploded view, the first diaphragm valve 2700 comprises a generally convex shape and comprises a circular protrusion 2710 near the apex of this convex shape. Similarly, the second diaphragm valve 2750 comprises a generally convex shape and comprises a circular protrusion 2760 near the apex of this convex shape.

The first diaphragm 2700 and/or the second diaphragm 2750 are made from a diaphragm material, which is a fluoroelastomer material or a perfluoroelastomer material or a mixture therefrom. In particular, the diaphragm material may be FKM ML-X18.

In a preferred arrangement of the dispense interface 2000, the manifold surface is positioned to reside along the generally flat surface 2040 of the inner body 2200. Preferably, in order to provide a seal between the manifold and the inner body 2200, these two components may be laser welded together.

In order to facilitate such a laser welding seal, in one arrangement, the inner body 2200 may be made from a cyclic olefin polymer material or a cyclic olefin copolymer material or a mixture therefrom which is preferably doped with a laser welding additive. Such a laser welding additive may increase the inner body's sensitivity to laser light. In addition, the manifold 2300 may be made from an optically clear cyclic olefin polymer or a cyclic olefin copolymer or a mixture therefrom so as to allow the welding laser to pass through the manifold 2300 and activate a mating surface area residing between the two components with minimal interference.

For instance, the surfaces 2304 and 2040 of the inner body 2200 and the manifold 2300, respectively, are joined at a joining area (e.g. the mating surface area and or a part of the mating surface area) defined by a laser welding track.

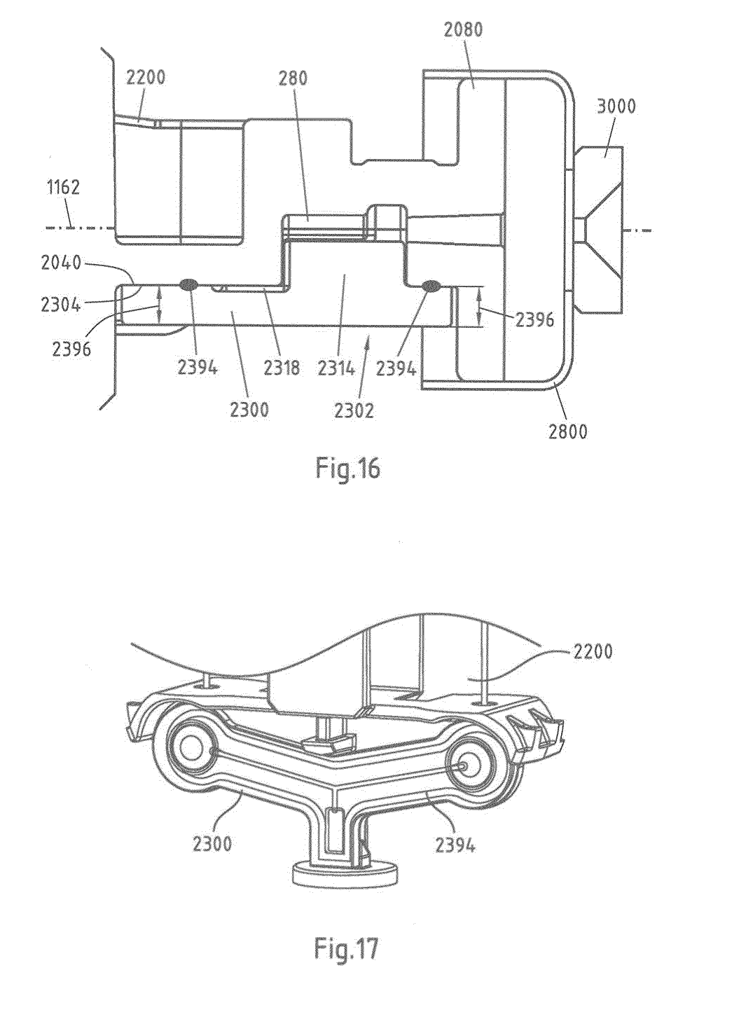

For example, FIGS. 16 and 17 illustrates the manifold 2300 provided along the flat surface of the inner body and then laser welded along a laser welding track 2394. As shown, this laser welding track 2394 extends along an outer edge of the manifold 2300. The large, flat mating surface area of the surfaces 2304, on the manifold and the inner body 2040 respectively, help to produce substantial surface areas for the welding to act upon and this tends to maximize the seal created between these two components.

In particular, the laser welding track 2394 is closed and extends along a substantially flat area of the surfaces 2304 and 2040 of the manifold and the inner body respectively. Furthermore, FIG. 16 illustrates a partial sectional view of the manifold 2300 laser welded to the inner body 2200. As illustrated in FIG. 16, the thickness 2396 of the manifold 2300 at the laser welding track 2394 is substantially uniform. This is inter alia advantageous to ensure a constant laser welding spot at the joining area defined by the laser welding track and a constant focal length of the welding laser.

If, as described above, the inner body 2200 and/or the manifold 2300 is made from a cyclic olefin polymer material or a cyclic olefin copolymer material or a mixture therefrom the inner body 2200 and/or the manifold 2300 provides a high dimensional stability. In particular, these materials are resistant to water so that dimensional instability due to water absorption does not occur. Furthermore, as the first diaphragm 2700 and/or the second diaphragm 2750 are made from the preferably plasticizer-free diaphragm material, the material of the inner body 2200 and/or the manifold 2300 cannot be softened by plasticizers leaking from the first and/or second diaphragm over time so that the inner body 2200 and/or the manifold 2300 may remain dimensionally stable for a long time. Other body parts as for example the main outer body 2100 may of course be made from a cyclic olefin polymer material or a cyclic olefin copolymer material or a mixture therefrom as well or instead. The advantages of these materials, in particular in combination with the diaphragms made of the diaphragm material, apply accordingly for these other body parts.

Preferably, the manifold 2300 further comprises a fluid groove arrangement 2318 and a rectangular protrusion or filling block 2314. As illustrated, referring to FIGS. 14 to 16, both the groove arrangement 2318 and the protrusion or filling block 2314 may be provided along a manifold top surface 2304. The protrusion 2314 may be provided near a distal end 2302 of the manifold 2300. In one preferred arrangement, this protrusion 2314 comprises a rectangular protrusion. With such a rectangular configuration, once the manifold 2300 is assembled (e.g., laser welded) along the flat surface 2040 of the inner body 2200, the protrusion 2314 will reside within the third cavity or holding chamber 280 of the inner body 2200. As illustrated, the rectangular protrusion or the filling block fills the majority of the third cavity or holding chamber while still redirecting fluid flow. One advantage of such a configuration is that it reduces the ullage of the dispense interface 2000. In addition, forming the fluid groove arrangement 2318 as a cavity between the two laser welded components allows the majority of the fluid groove geometry to be moulded using an open-and-shut tool. Consequently, use of an open-and-shut tool reduces the need for fragile core pins or split lines with the fluid groove arrangement. This also allows for relatively complex and tight tolerance geometry without complex tooling. The molding of key assembly snap features on the same component, such as a outer protrusion on the inner body 2200, also helps reduce tolerance stack-ups and also tends to allow for small needle wells and therefore smaller ullage.

In addition, the use of the needle guide 3000 to direct a Type A cannula means that the channel into which the cannula is received can be smaller as some of the tolerances on the needle position are reduced. The alignment of the flow path through the dispense interface also requires certain special considerations. In one example arrangement, both of the cartridges contained within the drug delivery device as well as the needle assembly are positioned in a single plane cutting through the depth of the drug delivery device along the longitudinal device centerline 1162. Furthermore, the longitudinal axis of the first and second piercing needles 240, 250 forming the inlet of the diaphragm valve 2700, 2750 and the first and second reservoir 2050, 2054 may be positioned in this single vertical plane. However, due to the positioning of the diaphragm valves 2700, 2750 and the fluid groove arrangement 2318 on one side of the dispense interface components, the fluid groove arrangement 2318 is moved off this centerline 1162. In particular, the diaphragm valves 2700, 2750 may be arranged such that they may provide a fluid seal between the first and second reservoir 2050, 2054, respectively, and the fluid groove arrangement 2318. Accordingly, the diaphragm valves 2700, 2750 may be arranged in another vertical plane spaced from and parallel to the single plane cutting through the depth of the drug delivery device along the longitudinal device centerline 1162. Also, the fluid groove arrangement 2318 forming the outlet of the diaphragm valves 2700, 2750 may be arranged in another single vertical plane spaced from and parallel to the single plane cutting through the depth of the drug delivery device along the longitudinal device centerline 1162.

The vertical arrangement of the diaphragm valves 2700, 2750 and the fluid groove arrangement 2318 in the dispense interface 2000 is inter alia advantageous to allow the manifold and the inner body to be joined by a laser positioned angled (e.g. perpendicular) to the first vertical plane cutting through the depth of the drug delivery device along the (longitudinal) device centerline 1162. Positioning the laser angled (e.g. perpendicular) to the first vertical plane is inter alia advantageous, because from such a horizontal position the laser may only need to pass through the manifold which may have an at least substantially uniform thickness at the laser welding track, whereas from a vertical position the laser may need to pass through additional components having no uniform thickness at the laser welding track. In other words, this vertical arrangement of the diaphragm valves 2700, 2750 and the fluid groove arrangement are inter alia advantageous to allow the first body part and the second body part to be joined by laser welding at a joining area of the vertically oriented surfaces 2304, 2040 which is easily accessible by a laser.

Prior to dispense through an attached needle assembly, the groove arrangement 2318 is brought back onto the centerline 1162 using the third cavity or holding chamber 280 molded into the inner body 2200. These factors combine to reduce the volume of liquid or medicament required to fill the dispense interface 1200 prior to dispense, thereby aiding dose accuracy.

Returning to the perspective view of the manifold 2300 provided by FIG. 14, preferably, the first valve cavity 2366 is positioned in the center of a first convex protrusion 2380 situated along the top surface 2304 of the manifold 2300. In such an arrangement, when the circular protrusion 2710 of the first diaphragm valve 2700 is seated within the first valve cavity 2366, the diaphragm valve 2700 provides a fluid seal between the first circular recess or reservoir 2050 defined by the inner body 2200 and the fluid groove arrangement 2318 provided along the top surface of the manifold 2300. However, if fluidic pressure is applied upon the first diaphragm valve 2700 (e.g., during a dose priming or a dose injecting step), the first valve 2700 will change from an un-stressed state to a stressed state. In the stressed state, fluidic pressure inverts the naturally convex shape of the first valve 2700 so that the convex nature of the first valve inverts and thereby will reside along a top surface of the first convex protrusion 2380. In this stressed condition, the first diaphragm valve 2700 will allow fluid to flow from the first reservoir of the inner body 2200 and the fluid groove arrangement 2318 of the manifold 2300.

Similarly, the second valve cavity 2372 is also shaped for receiving a circular protrusion 2760 of a second circular diaphragm valve 2750. Moreover, this second valve cavity 2372 is also positioned near an apex of a second convex protrusion 2390. The second diaphragm valve operates in a similar manner as the first diaphragm valve when fluid pressure is applied.

As will be explained in greater detail below, it is the operation of a first and second diaphragm valves 2700, 2750 along with a fluid groove arrangement 2318 that allows the first and second reservoirs 2050, 2054 of the inner body 2200 to be used for priming and dose administration of the first and/or second medicaments contained within a multiple medicament drug delivery device, such as the device illustrated in FIG. 1.

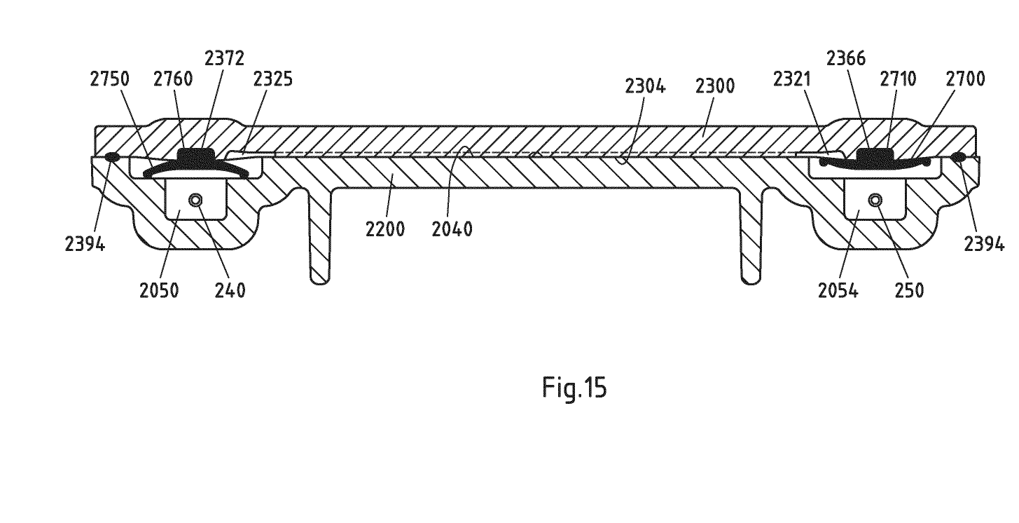

As described above, the presently disclosed dispense interface 2000 may comprise a valve arrangement comprising a first and a second diaphragm valve 2700, 2750. One advantage of utilizing such diaphragm or umbrella valves 2700, 2750 is that they characteristically tend to have low cracking or opening pressure. Another advantage of such valve structures is that they tend to provide low or minimal resistance to flow when open and they also tend to seal effectively against back pressure. These valves can also be designed to be very small in size, for example, on the order of approximately 3.5 mm to about approximately 4.5 mm. As such, these valves can tend to minimize the post valve ullage within the dispense interface 2000. However, other valve arrangements may also be utilized for the dispense interface 2000. In FIG. 15 a schematic cross-sectional view of the diaphragm valves 2700, 2750 arranged between the manifold 2300 and the inner body 2200 of the dispense interface 2000 is illustrated.

For example, a first fluid groove 2320 is provided along the manifold top surface 2304. This first fluid groove 2320 has a starting point 2321 near the first valve cavity 2366 but this first fluid groove 2320 is not in fluid communication with this first cavity. Similarly, a second fluid groove 2324 has a starting point 2325 near the second valve cavity 2372 but is not in fluid communication with this second cavity. As illustrated in FIG. 14, the first and second fluid grooves 2320, 2324 may be configured to meet near an intersection 2336 along the flat surface, near the middle of the T-shaped manifold 2300. At this intersection 2336, the first and second grooves 2320, 2324 meet at a third fluid groove 2328. This third groove 2328 resides in fluid communication with a fourth fluid groove 2332. In one preferred arrangement, this fourth fluid groove 2332 may be provided along an external surface of the rectangular protrusion 2314 provided along the bottom surface of the manifold 2300. As such, when the top surface 2304 of manifold 2300 is positioned along the generally flat surface 2040 of the inner body 2200 and then laser welded, the manifold 2300 and these plurality of fluid grooves 2320, 2324, 2328, and 2322 (i.e. fluid groove arrangement 2318) allow for fluid communication between the first and second reservoirs 2050, 2054 of the inner body 2200 and the holding chamber of the inner body 2200.

In addition, the substantially flat bottom surface of the manifold 2300 further comprises a first convex protrusion 2380 and a second convex protrusion 2390. Preferably, the first protrusion 2380 comprises a generally convex shape and further defines the first valve cavity 2366. Similarly, the second convex shaped protrusion defines the second valve cavity 2372. As will be described in greater detail below, when the top surface of the manifold 2300 is assembled along the flat surface of the inner body 2200, a first diaphragm valve protrusion is placed within this first circular shaped cavity and a second diaphragm valve protrusion will be placed within this second circular shaped cavity.

In the exemplary view of FIG. 15, the diaphragm valve 2700 is shown in a stressed state and the diaphragm valve 2750 is shown in a non-stressed state. Since the first and second diaphragm valves have a generally convex shape in a non-stressed position, in a non-stressed state, the convex nature of the diaphragm valve will provide a sealing arrangement between the manifold and the inner body so as to prevent any fluid from flowing from the first cavity of the inner body, through the first groove and into the holding chamber. However, in a stressed or non-steady state where pressure is exerted upon the convex diaphragm valves, the valve will come under stress and the unstressed convex nature of the diaphragm valve will be inverted, such that the valve will fold back towards the convex protrusion of the manifold. In this stressed position, the valve will therefore allow for fluid communication between the inner body first reservoir and the start portion of the first fluid groove which will then move towards the holding chamber by way of the third groove 2328 and also the fourth groove 2332 of the manifold. The second diaphragm valve operates in a similar manner to allow fluid to flow from the second reservoir of the inner body to the holding chamber of the inner body.

With the first diaphragm 2700 and/or the second diaphragm 2750 being made from a diaphragm material, which is a fluoroelastomer material or a perfluoroelastomer material or a mixture therefrom, the first diaphragm 2700 and/or the second diaphragm 2750 have suitable low compression set properties to return to their original shape after being in the stressed state. This ensures a reliable closing and a sufficient closing and sealing pressure of the first and/or second diaphragm valve over a long life-time. It is not necessary to add plasticizers to the diaphragm material to achieve these properties. Therefore, the diaphragm material maybe in particular plasticizer-free, so that a plasticizer contamination of the drug used with the apparatus can be prevented. Moreover, the diaphragm material is inert to insulin and shows good chemically resistance to m-cresol which is often used in insulin drugs.

The diaphragm material also shows inertness with body parts made of a cyclic olefin polymer material, a cyclic olefin copolymer material or a mixture therefrom, such as, for example, the inner body 2200 or the manifold 2300. These body parts remain dimensionally stable and do not soften as no plasticizer leaches from the first and/or the second diaphragm.

The preferred selection of a specific fluoroelastomer or perfluoroelastomer material, or a mixture therefrom used for a diaphragm and/or the preferred selection of a specific cyclic olefin polymer or cyclic olefin copolymer material, or a mixture therefrom used for a body part may depend on the drug which is to be used with the apparatus.

For example, some fluoroelastomer or perfluoroelastomer materials and some cyclic olefin polymer or cyclic olefin copolymer materials may show better and others less compatibility with certain drugs. However, the latter materials may show better compatibility with other drugs. The compatibility may be determined by experiment in each case.

In particular, a test series has been performed to determine the compatibility of fluoroelastomer or perfluoroelastomer materials, or mixtures therefrom, with insulin drugs. In this test series the (per)fluoroelastomer material FKM ML-X18 was found to have very good insulin compatibility.

A further test series has been performed to determine the compatibility of cyclic olefin polymer or cyclic olefin copolymer materials, or mixtures therefrom, with insulin drugs. In this test series the cyclic olefin polymer material Zeonor® 1020R provided by Zeon Europe GmbH was found to have very good insulin compatibility.

In said test series a first, a second and a third aqueous test solution have been used, which test solutions all have the following composition:

Insulin glargine: 3.64 mg/ml

Lixisenatid: 0.40 mg/ml

Zink Chloride: 30 μg/ml

L-Methionin: 3.0 mg/ml

Glycerol 85%: 20 mg/ml

m-Cresol: 2.7 mg/ml

NaOH/HCl 1N, ad pH 4.5

water for injection purposes ad 1 ml (ad 1.005 g)

A diaphragm made from FKM ML-X18 (provided by MiniVale) was added to the second test solution and a body part portion made from Zeonor 1020R was added to the third test solution. The first test solution was used as reference.

The impact of the diaphragm of body part material on the drug solution or on the second/third test solution, respectively, inter-alia depends on the ratio of surface of the material in contact with the solution. The surface to volume ratio of the diaphragm used in the test series was chosen to be A/V≈2.9 mm²/mm³ which is a realistic value for a diaphragm in an actual device. Therefore, the results of this test series should essentially reflect the expected results for an actual drug delivery device. The surface to volume ratio of the body part portion was chosen to be A/V≈1.5 mm²/mm³, which is lower than the realistic value of a complete body part but still may give a good estimation of the drug compatibility of Zeonor 1020R.

All three solutions were stored for a time of 14 days at a temperature of 25° C. and analyzed before the storing (0 days), after 2 days of storing, after 7 days of storing and after 14 days of storing at the end of the test series.

In order to determine the insulin compatibility of FKM ML-X18 and Zeonor 1020R, the first, the second and the third test solution were analyzed by high performance liquid chromatography (HPLC), more specifically size exclusion chromatography, and different parameters were determined according to the following tests 1 to 6:

Test 1: Insulin glargine assay to determine the concentration of insulin glargine in the test solutions;

Test 2: M-cresol assay to determine the concentration of M-cresol in the test solutions;

Test 3: Determination of the sum amount of impurities related to lixisenatide (AVE0010), i.e. degradation products of lixisenatide;

Test 4: Determination of the sum amount of impurities related to insulin glargine, i.e. degradation products of insulin glargine;

Test 5: Determination of the fraction of HMWPs (high molecular protein weights) for insulin glargine;

Test 6: Determination of the pH-value.

Table 1 below shows the results of the analysis. The first column refers to the test number as described above. The second column refers to the analyzed test solution, wherein the first test solution is referred to as “Reference”, the second test solution is referred to as “FKM ML-X18” and the third test solution is referred to as “Zeonor 1020R”.

The results for tests 1 and 2 are given in percent in relation to the original amount of insulin glargine and m-cresol, respectively. The results for tests 3 and 4 are given in percent in relation to the total amount of the component under study, i.e. lixisenatide or insulin glargine, respectively. The results for test 5 are given in area percent in relation to the total amount of insulin glargine. A result in area percent was calculated by dividing the area of the HWPS signal by the sum of the area of the total amount of insulin glargine.

TABLE 1

| | | 0 days | 2 days | 7 days | 14 days |
|---|---|---|---|---|---|
| Test 1 | Reference | 98.6% | 97.5% | 98.4% | 98.1% |
| | FKM ML-X18 | 98.1% | 97.3% | 97.3% | 98.1% |
| | Zeonor 1020R | 101.1% | — | — | 98.1% |
| Test 2 | Reference | 97.4% | 97.8% | 99.6% | 96.7% |
| | FKM ML-X18 | 95.2% | 89.6% | 82.2% | 78.5% |
| | Zeonor 1020R | 100.0% | — | — | 95.9% |
| Test 3 | Reference | 1.3% | 1.2% | 1.4% | 1.3% |
| | FKM ML-X18 | 0.6% | 1.2% | 1.9% | 2.9% |
| | Zeonor 1020R | 0.8% | — | — | 2.4% |
| Test 4 | Reference | 0.2% | 0.2% | 0.2% | 0.2% |
| | FKM ML-X18 | 0.2% | 0.2% | 0.5% | 0.8% |
| | Zeonor 1020R | 0.3% | — | — | 0.6% |
| Test 5 | Reference | 0.05% | 0.06% | 0.06% | 0.10% |
| | FKM ML-X18 | 0.04% | 0.07% | 0.15% | 0.20% |
| | Zeonor 1020R | 0.1% | — | — | 0.1% |
| Test 6 | Reference | pH 4.6 | pH 4.6 | pH 4.5 | pH 4.6 |
| | FKM ML-X18 | pH 4.7 | pH 4.5 | pH 4.0 | pH 3.8 |
| | Zeonor 1020R | pH 4.5 | — | — | pH 4.6 |

According to the results of test 1 the solution with FKM ML-X18 and the solution with Zeonor 1020R showed no significant decrease in the insulin glargine assay even after 14 days of storage at 25° C. A degradation of insulin glargine was also not observed for FKM ML-X18 and Zeonor 1020R.

The increase in high molecular weight proteins (test 5) was at a moderate and acceptable level for both materials.

M-cresol is used in the formulation as a preservative. For the solution with FKM ML-X18 (test 2), a 20% decrease of the m-cresol assay was observed after 14 days, i.e. the FKM ML-X18 has absorbed part of the m-cresol. However, a decrease of 20% after 14 days is an acceptable value and in particular this value is better than for many other elastomers. The M-cresol absorption of Zeonor 1020R was found to be very low.

The increase of impurities related to lixisenatide (AVE0010) (test 3) and insulin glargine (test 4) for FKM ML-X18 proved to be very moderate in comparison with other elastomers. Similarly, the impurity level for Zeonor 1020R was low as well.

The insulin glargine is formulated at an acidic pH 4, where it is completely water soluble. After subcutaneous injection of the acidic solute, when a physiologic pH (approximately 7.4) is achieved, the increase in pH causes the insulin to come out of solution resulting in the formation of higher order aggregates of insulin hexamers. Because of this, it is very important that the pH does not change into the alkaline direction before injection as insulin glargine then would already precipitate in the solution.

In the test of FKM ML-X18 the pH value changed to a slightly more acidic regime during the course of the test series. No significant adverse impact on the insulin glargine or lixiseatide was found due to this acidic change. Advantageously, the second test solution with FKM ML-X18 did not show a pH value change to an alkaline regime which could lead to precipitation of insulin glargine as described above.

In conclusion, the solution with FKM ML-X18 did not show any significant degradation after 14 days of storage at 25° C. FKM ML-X18 therefore proved to have a high compatibility for insulin drugs and is therefore a preferred material to be used for diaphragms of diaphragm valves in medical devices designated for insulin drugs.

Similarly, also Zeonor 1020R proved to have high compatibility with insulin drugs so that it is a preferred material to be used for body parts in medical devices designated for insulin drugs.

Further tests were carried out to analyze material compatibility of cyclic olefin (co)polymer materials and (per)fluoroelastomer materials:

An inner body, a manifold and a diaphragm like the inner body 2200, the manifold 2300 and the diaphragm 2700 described above with reference to FIGS. 12 to 17 were assembled as shown in FIG. 15, though the inner body and the manifold were only clamped instead of permanently joined. The inner body and the manifold were made from a perfluoroelastomer material (ML952 Rezlyn pale) and the diaphragm was made from a cyclic olefin polymer material (Zeonor 1020R). This assembly was then stored for 186 hours at a temperature of 60° C. to accelerate the testing. After the storing, the assembly was disassembled and the contact surfaces of the diaphragm with the inner body and the manifold were analyzed by optical and tactile inspection.

It was found that the contact surfaces did not show any mechanical degradation due to material incompatibility. Therefore, the cyclic olefin polymer material proved to be compatible with the perfluoroelastomer material.

In an according test with a diaphragm valve made from a fluoroelastomer material (ML652 lilac) the contact surfaces also showed no deformation after the storing. Although the contact surface appeared to be slightly sticky in this test, the compatibility on the whole still proved to be acceptable.

As a comparative example, the same test was carried out with an inner body and a manifold made from a cyclic olefin polymer material (Zeonor 1020R) and a diaphragm made from a thermoplastic elastomer (Kraiburg TM5/6/7MED). After the storing under the same conditions as described above the contact surfaces of the diaphragm with the inner body and the manifold showed considerable deformations. The combination of these two materials therefore could lead to leakages and to a reduced lifetime of the diaphragm valve.

Further tests were carried out to analyze the suitability of (per)fluoroelastomer materials for use in diaphragm valves. In an assembly as shown in the context of FIG. 15, the diaphragm valve is assembled in a pretension condition. This is necessary as the diaphragm valve is a passive valve and to ensure that the valve creates a minimum of sealing pressure even in a non-pressurized state of the apparatus.

Elastic materials—as for the diaphragms—tend to set over time and temperature. This so-called “compression-set” is a permanent deformation of the elastic material and thus is a loss of sealing pressure and capability.

To analyze the compression-set of a diaphragm made from a fluoroelastomer material or a perfluoroelastomer material or a mixture therefrom, diaphragms like the diaphragm 2700 shown in FIG. 15 and made from a perfluoroelastomer material (FFKM-ML952) and from a fluoroelastomer material (FKM-ML-X18) were placed in an aluminium mock-up of the inner body and manifold as shown in FIG. 15. The material aluminium was chosen to not affect the results by any possible material interaction between the valves and the inner body and the manifold.

The nominal height of the diaphragms was 1.25 mm. For the tests, the diaphragms were compressed to three different compression heights (0.95 mm, 0.85 mm, 0.75 mm). These compression heights were chosen to represent different tolerance conditions possible in the real apparatus. The tests were performed for 186 h at a temperature of 60° C. The heights of the diaphragms were measured before and after the test. The difference between two according measurements is considered as a value for the compression-set of the respective diaphragm. The lower the compression-set the better are the resealing capabilities of the diaphragm.

The diaphragm made from the perfluoroelastomer material only showed a compression-set of less than about 0.07 mm for all three compression heights. Likewise, the diaphragm made from the fluoroelastomer material only showed a compression-set of less than about 0.09 mm for all three compression heights. These results prove that diaphragms made from (per)fluoroelastomer materials offer a very good long-time resealing capabilities.

A comparable test was also carried out for diaphragms made from thermoplastic elastomers (Kraiburg TM5/6/7MED) which showed compression-sets of between 0.25 and 0.4 mm for the compression height 0.75 mm. Thus, these materials have less resealing capabilities than the (per)fluoroelastomer materials.

Further tests were carried out to analyze the laser weldability of cyclic olefin (co)polymer materials Inner bodies and manifolds made from a cyclic olefin polymer material (Zeon1020R) were laser welded. Some of the joints were in a natural state for welding, while other joints were enriched with black additives to improve laser light absorption. The joints were welded with a 1060 nm wavelength direct diode laser.

The welded assemblies were then pressurized by 45 N on a 1 ml cartridge for 20 s, which equals the pressure application of 12.2 bar, and the tightness of the assemblies was determined. The majority of the laser welded assemblies (90%) passed this leakage test. Thus, cyclic olefin (co)polymer materials proved to have good laser weldability.

In conclusion, the tests described above show that the combination of a fluoroelastomer material or a perfluoroelastomer material or a mixture therefrom for a diaphragm and a cyclic olefin polymer material or a cyclic olefin copolymer material or a mixture therefrom for a body part yields apparatuses with long life-time and good drug compatibility, especially for insulin glargine drugs.

The present disclosure also comprises the following embodiments:

1. An apparatus comprising: a body part, wherein the body part is made from a high-density polyethylene (HDPE) material, and a diaphragm valve with a diaphragm, wherein at least a surface portion of the diaphragm is made from a diaphragm material and wherein said diaphragm material is a thermoplastic elastomer (TPE) material.

2. An apparatus according to embodiment 1 described above, wherein said diaphragm material has a content of plasticizers of less than 10% by weight, preferably of less than 5% by weight, more preferably of less than 1% by weight, especially of less than 0.5% by weight.

3. An apparatus according to embodiment 1 or 2 described above, wherein said diaphragm material is a plasticizer-free thermoplastic elastomer material.

4. An apparatus according to any one of embodiments 1 to 3 described above, wherein the diaphragm is entirely made from said diaphragm material.

5. An apparatus according to any of embodiments 1 to 4 described above, wherein the diaphragm valve is accommodated within a housing which is at least partially formed by the body part.

6. An apparatus according to any of the embodiments 1 to 5 described above, wherein the body part is in direct contact with the diaphragm.

7. An apparatus according to any of the embodiments 1 to 6 described above, the apparatus comprising: a first body part; and a second body part configured as cover part, wherein said first body part and said second body part are configured to at least partially form a fluid channel between a surface of said first body part and a surface of said second body part, and wherein the first and/or the second body part is/are made from a high-density polyethylene material.

8. An apparatus according to embodiment 7 described above, said first body part comprising: a recess; and a first body part reservoir and/or a second body part reservoir, wherein said fluid channel provides a fluid connection from said first body part reservoir and/or said second body part reservoir to said recess, and wherein at least one of said first and/or second body part reservoir is configured to receive the diaphragm valve.

9. An apparatus according to embodiments 7 or 8 described above, wherein said first body part defines an inner body of said apparatus and said second body part defines a manifold of said apparatus.

10. An apparatus according to any of the embodiments 7 to 9 described above, wherein said second body part comprises at least a first valve cavity provided along its top surface, wherein the first valve cavity is shaped for receiving a protrusion of the diaphragm of the diaphragm valve and wherein the first valve cavity is positioned near an apex of the protrusion of the diaphragm.

11. An apparatus according to any of the embodiments 7 to 10 described above, wherein said surfaces of said first body part and said second body part are joined by laser welding at a joining area.

12. An apparatus according to any of the embodiments 1 to 11 described above, wherein the apparatus is a medical device or a part of a medical device.

13. An apparatus according to any of the embodiments 1 to 12 described above, wherein said diaphragm valve is configured to enable fluid flow, if a fluidic pressure threshold is applied on said diaphragm valve.

14. An apparatus according to embodiment 12 described above, wherein the apparatus is a medical device configured to eject a medicament and wherein the diaphragm valve is configured to control fluid communication of a medicament contained in a reservoir of the drug delivery device and a dose dispenser.

15. An apparatus according to embodiment 14 described above, wherein said apparatus comprises at least two of said diaphragm valves and the medical device respectively comprises at least two of said reservoirs.

These embodiments 1 to 15 offer similar advantages as the embodiments of the apparatuses described before, which comprise a body part made from a cyclic olefin polymer material or a cyclic olefin copolymer material or a mixture therefrom, and a diaphragm valve with a diaphragm, wherein at least a surface portion of the diaphragm is made from a diaphragm material and wherein said diaphragm material is a fluoroelastomer material or a perfluoroelastomer material or a mixture therefrom. Regarding the advantages and further features of the embodiments 1 to 15 it is therefore referred to the description of the embodiments of the apparatuses described before.

Furthermore, it is an advantage of the apparatus according to embodiment 1 that use of a high-density polyethylene material and a thermoplastic elastomer material allows for more economic production due to cheaper material costs.

A test series has been performed to determine the compatibility of thermoplastic elastomer materials with insulin drugs. In this test series the thermoplastic elastomer material THERMOPLAST® TM6MED provided by KRAIBURG TPE GmbH & Co.KG was found to have very good insulin compatibility.

A further test series has been performed to determine the compatibility of high-density polyethylene material with insulin drugs. In this test series the high-density polyethylene material SABIC® PCG80063 provided by SABIC Deutschland GmbH was found to have very good insulin compatibility.

In said test series a first, a second and a third aqueous test solution have been used, which test solutions all have the following composition:

Insulin glargine: 3.64 mg/ml

Lixisenatid: 0.40 mg/ml

Zink Chloride: 30 μg/ml

L-Methionin: 3.0 mg/ml

Glycerol 85%: 20 mg/ml

m-Cresol: 2.7 mg/ml

NaOH/HCl 1N, ad pH 4.5

water for injection purposes ad 1 ml (ad 1.005 g)

A diaphragm made from TPE TM6MED (provided by MiniVale) was added to the second test solution and a body part portion made from PCG80063 was added to the third test solution. The first test solution was used as reference.

The impact of the diaphragm material on the drug solution or on the second test solution, respectively, inter-alia depends on the ratio of surface of the material in contact with the solution. The surface to volume ratio of the diaphragm used in the test series was chosen to be A/V≈2.9 mm²/mm³ which is a realistic value for a diaphragm in an actual device. Therefore, the results of this test series should essentially reflect the expected results for an actual drug delivery device. The surface to volume ratio of the body part portion was chosen to be A/V≈1.5 mm²/mm³ which is lower than the value of a complete body part but still may give a good estimation of the drug compatibility of PCG80063.

All three solutions were stored for a time of 14 days at a temperature of 25° C. and analyzed before the storing (0 days), after 2 days of storing, after 7 days of storing and after 14 days of storing at the end of the test series.

In order to determine the insulin compatibility of TPE TM6MED and PCG80063, the first, the second and the third test solution were analyzed by high performance liquid chromatography (HPLC), more specifically size exclusion chromatography, and different parameters were determined according to the following tests 1 to 6:

Test 1: Insulin glargine assay to determine the concentration of insulin glargine in the test solutions;

Test 2: M-cresol assay to determine the concentration of M-cresol in the test solutions;

Test 3: Determination of the sum amount of impurities related to lixisenatide (AVE0010), i.e. degradation products of lixisenatide;

Test 4: Determination of the sum amount of impurities related to insulin glargine, i.e. degradation products of insulin glargine;

Test 5: Determination of the fraction of HMWPs (high molecular protein weights) for insulin glargine;

Test 6: Determination of the pH-value.

Table 2 below shows the results of the analysis. The first column refers to the test number as described above. The second column refers to the analyzed test solution, wherein the first test solution is referred to as “Reference”, the second test solution is referred to as “TPE TM6MED” and the third test solution is referred to as “PCG80063”.

The results for tests 1 and 2 are given in percent in relation to the original amount of insulin glargine and m-cresol, respectively. The results for tests 3 and 4 are given in percent in relation to the total amount of the component under study, i.e. lixisenatide or insulin glargine, respectively. The results for test 5 are given in area percent in relation to the total amount of insulin glargine. A result in area percent was calculated by dividing the area of the HWPS signal by the sum of the area of the total amount of insulin glargine.

TABLE 2

| | | 0 days | 2 days | 7 days | 14 days |
|---|---|---|---|---|---|
| Test 1 | Reference | 98.9% | 100.3% | 98.1% | 98.6% |
| | TPE TM6MED | 98.4% | 98.6% | 98.6% | 95.6% |
| | PCG80063 | 102.5% | — | 97.8% | 95.3% |
| Test 2 | Reference | 97.8% | 98.8% | 97.0% | 97.0% |
| | TPE TM6MED | 82.6% | 81.1% | 80.0% | 77.4% |
| | PCG80063 | 101.1% | — | 94.4% | 91.5% |
| Test 3 | Reference | 1.2% | 1.0% | 1.0% | 2.0% |
| | TPE TM6MED | 1.1% | 1.5% | 2.7% | 4.1% |
| | PCG80063 | 1.2% | — | 1.5% | 2.2% |
| Test 4 | Reference | 0.3% | 0.2% | 0.3% | 0.4% |
| | TPE TM6MED | 0.2% | 0.3% | 0.7% | 1.0% |
| | PCG80063 | 0.4% | — | 0.6% | 0.6% |
| Test 5 | Reference | 0.05% | 0.04% | 0.04% | 0.06% |
| | TPE TM6MED | 0.08% | 0.06% | 0.17% | 0.30% |
| | PCG80063 | 0.07% | — | 0.09% | 0.12% |
| Test 6 | Reference | pH 4.6 | pH 4.6 | pH 4.6 | pH 4.5 |
| | TPE TM6MED | pH 4.5 | pH 4.5 | pH 4.6 | pH 4.6 |
| | PCG80063 | pH 4.6 | — | pH 4.5 | pH 4.6 |

Regarding the detailed assessment of these test results presented in table 2, it is referred to the discussion of the test results from table 1.

In conclusion, TPE TM6MED proved to have high compatibility for insulin drugs and is therefore a preferred material to be used for diaphragms of diaphragm valves in medical devices designated for insulin drugs.

Similarly, also SABIC® PCG80063 proved to have high compatibility with insulin drugs so that it is a preferred material to be used for body parts in medical devices designated for insulin drugs.

Further tests were carried out to analyze material compatibility of thermoplastic elastomer materials:

An inner body, a manifold and a diaphragm like the inner body 2200, the manifold 2300 and the diaphragm 2700 described above with reference to FIGS. 12 to 17 were assembled as shown in FIG. 15, though the inner body and the manifold were only clamped instead of permanently joined. The inner body and the manifold were made from a thermoplastic elastomer material (TPE TM6MED) and the diaphragm was made from a high-density polyethylene material (PCG80063). This assembly was then stored for 186 hours at a temperature of 60° C. to accelerate the testing. After the storing, the assembly was disassembled and the contact surfaces of the diaphragm with the inner body and the manifold were analyzed by optical and tactile inspection.

It was found that the contact surfaces showed slight residues due to the contact of the TPE TM6MED with the PCG80063 material. The contact surfaces however did not show any deformation after the storing. Therefore, the compatibility of the two materials on the whole proved to be good.

Further tests were carried out to analyze the suitability of thermoplastic elastomer materials for use in diaphragm valves. In an assembly as shown in the context of FIG. 15, the diaphragm valve is assembled in a pretension condition. This is necessary as the diaphragm valve is a passive valve and to ensure that the valve creates a minimum of sealing pressure even in a non-pressurized state of the apparatus.

Elastic materials—as for the diaphragms—tend to set over time and temperature. This so-called “compression-set” is a permanent deformation of the elastic material and thus is a loss of sealing pressure and capability.

To analyze the compression-set of a diaphragm made from thermoplastic elastomer material, diaphragms like the diaphragm 2700 shown in FIG. 15 and made from a thermoplastic elastomer material (TPE TM6MED) were placed in an aluminium mock-up of the inner body and manifold as shown in FIG. 15. The material aluminium was chosen to not affect the results by any possible material interaction between the valves and the inner body and the manifold.

The nominal height of the diaphragms was 1.25 mm. For the tests, the diaphragms were compressed to three different compression heights (0.95 mm, 0.85 mm, 0.75 mm). These compression heights were chosen to represent different tolerance conditions possible in the real apparatus. The tests were performed for 186 h at a temperature of 60° C. The heights of the diaphragms were measured before and after the test. The difference between two according measurements is considered as a value for the compression-set of the respective diaphragm. The lower the compression-set the better are the resealing capabilities of the diaphragm.

The diaphragms made from the thermoplastic elastomer material showed a compression-set of between about 0.11 mm and 0.17 mm for a compression height of 0.95 mm, a compression-set of between about 0.22 and 0.25 mm for a compression height of 0.85 mm and a compression-set of between about 0.31 mm and 0.35 mm for a compression height of 0.75 mm.

These results show that diaphragms made from thermoplastic elastomer materials offer long-time resealing capabilities which are worse than for (per)fluoroelastomer materials but still at a satisfactory level.

Further tests were carried out to analyze the laser weldability of high-density polyethylene materials. Inner bodies and manifolds made from a high-density polyethylene material (PCG80063) were laser welded. Some of the joints were in a natural state for welding, while other joints were enriched with black additives to improve laser light absorption. The joints were welded with a 1060 nm wavelength direct diode laser.

The welded assemblies were then pressurized by 45 N on a 1 ml cartridge for 20 s, which equals the pressure application of 12.2 bar, and the tightness of the assemblies was determined. The majority of the laser welded assemblies passed this leakage test. Thus, high-density polyethylene materials proved to have sufficient laser weldability.

In conclusion, the tests described above show that the combination of a thermoplastic elastomer material for a diaphragm and a high-density polyethylene material (PCG80063) for a body part yields apparatuses with sufficiently long life-time and sufficient drug compatibility, especially for insulin glargine drugs.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-15. (canceled)

16. Apparatus comprising:
a body part, wherein the body part is made from a cyclic olefin polymer material or a cyclic olefin copolymer material or a mixture therefrom, and
a diaphragm valve with a diaphragm, wherein at least a surface portion of the diaphragm is made from a diaphragm material and wherein said diaphragm material is a fluoroelastomer material or a perfluoroelastomer material or a mixture therefrom.

17. Apparatus according to claim 16,
wherein said diaphragm material has a content of plasticizers of less than 10% by weight, preferably of less than 5% by weight, more preferably of less than 1% by weight, especially of less than 0.5% by weight.

18. Apparatus according to claim 16,
wherein said diaphragm material is a plasticizer-free fluoroelastomer material or a plasticizer-free perfluoroelastomer material or a mixture therefrom.

19. Apparatus according to claim 16,
wherein the diaphragm is entirely made from said diaphragm material.

20. Apparatus according to claim 16,
wherein the diaphragm valve is accommodated within a housing which is at least partially formed by the body part.

21. Apparatus according to claim 16,
wherein the body part is in direct contact with the diaphragm.

22. Apparatus according to claim 16, the apparatus comprising:
a first body part; and
a second body part configured as cover part,
wherein said first body part and said second body part are configured to at least partially form a fluid channel between a surface of said first body part and a surface of said second body part, and
wherein the first and/or the second body part is/are made from a cyclic olefin polymer material or a cyclic olefin copolymer material or a mixture therefrom.

23. Apparatus according to claim 22, said first body part comprising:
a recess; and
a first body part reservoir and/or a second body part reservoir,
wherein said fluid channel provides a fluid connection from said first body part reservoir and/or said second body part reservoir to said recess, and
wherein at least one of said first and/or second body part reservoir is configured to receive the diaphragm valve.

24. Apparatus according to claim 22,
wherein said first body part defines an inner body of said apparatus and said second body part defines a manifold of said apparatus.

25. Apparatus according to claim 22,
wherein said second body part comprises at least a first valve cavity provided along its top surface,
wherein the first valve cavity is shaped for receiving a protrusion of the diaphragm of the diaphragm valve and
wherein the first valve cavity is positioned near an apex of the protrusion of the diaphragm.

26. Apparatus according to claim 22,
wherein said surfaces of said first body part and said second body part (2054) are joined by laser welding at a joining area.

27. Apparatus according to claim 16,
wherein the apparatus is a medical device or a part of a medical device.

28. Apparatus according to claim 16,
wherein said diaphragm valve is configured to enable fluid flow, if a fluidic pressure threshold is applied on said diaphragm valve.

29. Apparatus according to claim 27,
wherein the apparatus is a medical device configured to eject a medicament and
wherein the diaphragm valve is configured to control fluid communication of a medicament contained in a reservoir of the drug delivery device and a dose dispenser.

30. Apparatus according to claim 29,
wherein said apparatus comprises at least two of said diaphragm valves and the medical device respectively comprises at least two of said reservoirs.
